(19) United States
(12) Patent Application Publication
(10) Pub. No.: US 2024/0327426 A1
(43) Pub. Date: Oct. 3, 2024

(54) CRYSTALLINE FORMS OF CEFTIBUTEN

(71) Applicant: QPEX BIOPHARMA, INC., San Diego, CA (US)

(72) Inventors: Scott J. Hecker, San Diego, CA (US); David Jonaitis, San Diego, CA (US)

(21) Appl. No.: 18/574,702

(22) PCT Filed: Jun. 17, 2022
(86) PCT No.: PCT/US2022/073019

§ 371 Date: Dec. 27, 2023

Related U.S. Application Data
(60) Provisional application No. 63/217,710, filed on Jul. 1, 2021.

(51) Int. Cl. C07D 501/22; A61K 31/546; A61K 45/06

(52) U.S. Cl. CPC C07D 501/22; A61K 31/546; A61K 45/06

(57) ABSTRACT

Disclosed herein are crystalline forms of ceftibuten, methods of making such crystalline forms, and methods of using such crystalline forms. The crystalline forms of ceftibuten may be used in combination with β-lactamase inhibitors for the treatment of bacterial infections.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims the benefit of U.S. provisional application No. 63/217,710, filed Jul. 1, 2021, which is incorporated in its entirety.

### STATEMENT REGARDING FEDERALLY SPONSORED R&D

This invention was made with U.S. government support under the Department of Health and Human Services Contract No. HHSO100201600026C. The U.S. government has certain rights in the invention.

### BACKGROUND OF THE INVENTION

### Field of the Invention

The present application relates to the fields of pharmaceutical chemistry, biochemistry, and medicine. In particular, it relates to crystalline forms of ceftibuten and methods of making and using the same.

### Description of the Related Art

Ceftibuten is a third-generation cephalosporin antibiotic and is used to treat a variety of infections such as pharyngitis, tonsilitis, and pneumonia. There is a need for improved formulations of ceftibuten, particularly for use with β-lactamase inhibitors.

### SUMMARY

Some embodiments provide a crystalline form of a compound of Formula (I):

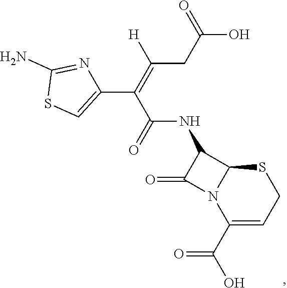

or a solvate thereof.

In some embodiments, the crystalline form of a compound of Formula (I) may exhibit an X-ray powder diffraction pattern comprising at least one characteristic peak selected from the group consisting of approximately 6.3, 9.9, 10.6, 12.5, 15.2, 18.8, 20.3, 21.0, 21.3, 21.4, 26.1, 26.3, and 30.0 degrees 2θ. In some embodiments, the crystalline form of the compound of Formula (I) may exhibit an X-ray powder diffraction pattern comprising at least three characteristic peaks, wherein the characteristic peaks are selected from the group consisting of 6.3, 9.9, 10.6, 12.5, 15.2, 18.8, 20.3, 21.0, 21.3, 21.4, 26.1, 26.3, and 30.0 degrees 2θ.

In some embodiments, the crystalline form of the compound of Formula (I) may exhibit an X-ray powder diffraction pattern comprising at least one characteristic peak selected from the group consisting of approximately of 6.4, 8.0, 10.0, 12.8, 13.1, 15.5, 16.1, 17.0, 19.1, 19.3, 20.5, 22.1, 22.5, 23.5, 25.0, and 26.4 degrees 2θ. In some embodiments, the crystalline form of the compound of Formula (I) may exhibit an X-ray powder diffraction pattern comprising at least three characteristic peaks selected from the group consisting of approximately of 6.4, 8.0, 10.0, 12.8, 13.1, 15.5, 16.1, 17.0, 19.1, 19.3, 20.5, 22.1, 22.5, 23.5, 25.0, and 26.4 degrees 2θ.

Additional embodiments provided herein include compositions comprising a crystalline form of the compound of Formula (I). In some embodiments, the total weight of the compound of Formula (I) in the composition may comprise greater than 50% by weight of the crystalline form. In some embodiments, the total weight of the compound of Formula (I) in the composition may comprise greater than 85% by weight of the crystalline form. In some embodiments, the total weight of the compound of Formula (I) in the composition may comprise greater than 90% by weight of the crystalline form. In some embodiments, the total weight of the compound of Formula (I) in the composition may comprise essentially of the crystalline form.

Additional embodiments provided herein include a method of treating a bacterial infection comprising administering to the subject a therapeutically effective amount of the crystalline form of the compound of Formula (I). In some embodiments, the disease or disorder may be selected from the group consisting of acute bacterial exacerbations of chronic bronchitis; acute bacterial otitis media; pharyngitis; tonsilitis; pneumonia; urinary tract infection; enteritis; and gastroenteritis. In some embodiments, the method of treatment may further comprise administering a β-lactamase inhibitor.

Further embodiments provided herein include a pharmaceutical composition, comprising a therapeutically effective amount of the crystalline form of the compound of Formula (I) and one or more pharmaceutically acceptable excipients. In some embodiments, the pharmaceutical composition further includes a β-lactamase inhibitors.

Further embodiments provided herein include methods of making the crystalline forms of the compound of Formula (I). In some embodiments, the method includes drying the compound under vacuum and nitrogen flow. In some embodiments, the method includes drying the compound under vacuum without flowing nitrogen over the compound.

### BRIEF DESCRIPTION OF THE DRAWINGS

FIG. 1 is an X-ray powder diffraction pattern of a crystalline form of the compound of Formula (I).

FIG. 2 is a table of the peak data of the X-ray powder diffraction pattern of FIG. 1.

FIG. 3 is an infrared spectrum of the crystalline form of FIG. 1.

FIG. 4 is a table of the peak data of the spectrum of FIG. 3.

FIG. 5 is a Raman spectrum of the crystalline form of FIG. 1.

FIG. 6 is a table of the peak data of the spectrum of FIG. 4.

FIG. 7 is a low-frequency Raman spectrum of the crystalline form of FIG. 1.

FIG. 8 is a table of the peak data of the spectrum of FIG. 7.

FIG. 9 is a solid-state NMR spectrum of the crystalline form of FIG. 1.

FIG. 10 is a table of the peak data of the spectrum of FIG. 9.

FIG. 11 is an X-ray powder diffraction pattern of a crystalline form of the compound of Formula (I).

FIG. 12 is a table of the peak data of the X-ray powder diffraction pattern of FIG. 11.

FIG. 13 is an infrared spectrum of the crystalline form of FIG. 11.

FIG. 14 is a table of the peak data of the spectrum of FIG. 13.

FIG. 15 is a Raman spectrum of the crystalline form of FIG. 11.

FIG. 16 is a table of the peak data of the spectrum of FIG. 14.

FIG. 17 is a low-frequency Raman spectrum of the crystalline form of FIG. 11.

FIG. 18 is a table of the peak data of the spectrum of FIG. 17.

FIG. 19 is a solid-state NMR spectrum of the crystalline form of FIG. 11.

FIG. 20 is a table of the peak data of the spectrum of FIG. 19.

FIG. 21 is an X-ray powder diffraction pattern of a crystalline form of the compound of Formula (I).

FIG. 22 is an X-ray powder diffraction pattern of a crystalline form of the compound of Formula (I).

FIG. 23 shows X-ray powder diffraction patterns of a crystalline form of the compound of Formula (I) in a relative humidity experiment.

FIG. 24 is a moisture sorption-desorption plot of the crystalline form of FIG. 1.

FIG. 25 is a moisture sorption-desorption plot of the crystalline form of FIG. 11.

FIG. 26 is a differential scanning calorimetry thermogram of the crystalline form of FIG. 1.

FIG. 27 is a differential scanning calorimetry thermogram of the crystalline form of FIG. 11.

### DETAILED DESCRIPTION

Disclosed herein are crystalline forms of ceftibuten, or solvates thereof, methods of crystallizing the crystalline forms of ceftibuten, as well as methods of treatment through administration of ceftibuten. Ceftibuten has the structure of Formula (I), shown below:

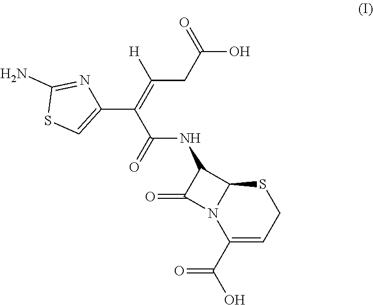

The crystalline forms described herein may be used in formulations for treating bacterial infections. In some embodiments, the crystalline forms are co-administered with a beta-lactamase inhibitor, either in the same formulation or separate formulations.

### Definitions

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula-OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkylthio” refers to the formula-SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2, -dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atom, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

As used herein, “alkylene” means a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” means a straight or branched chain di-radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, Nor S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is selected from hydrogen, optionally substituted C₁₋₆ alkyl, halogen, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, optionally substituted C₁₋₆ alkyl, halogen, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein.

A “C-carboxy” group refers to a “—C(═O) OR” group in which R is selected from hydrogen, halogen, optionally substituted C₁₋₆ alkyl, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein. A non-limiting example includes carboxyl (i.e., —C(═O) OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, optionally substituted C₁₋₆ alkyl, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein.

A “sulfonyl” group refers to an “—SO₂R” group in which R is selected from hydrogen, optionally substituted C₁₋₆ alkyl, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein.

An “S-sulfonamido” group refers to a “—SO₂NR_{A}R_{B}” group in which R_{A} and R_{B} are each independently selected from hydrogen, halogen, optionally substituted C₁₋₆ alkyl, optionally substituted C₁₋₆ alkoxy, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein.

An “N-sulfonamido” group refers to a “—N(R_{A}) SO₂R_{B}” group in which R_{A} and R_{B} are each independently selected from hydrogen, halogen, optionally substituted C₁₋₆ alkyl, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein.

A “C-amido” group refers to a “—C(═O)NR_{A}R_{B}” group in which R_{A} and R_{B} are each independently selected from hydrogen, halogen, optionally substituted C₁₋₆ alkyl, optionally substituted C₁₋₆ alkoxy, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein.

An “N-amido” group refers to a “—N(R_{A})C(═O)R_{B}” group in which R_{A} and R_{B} are each independently selected from hydrogen, halogen, optionally substituted C₁₋₆ alkyl, optionally substituted C₁₋₆ alkoxy, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S) NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, aC₆₋₁₀ ryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, halogen, optionally substituted C₁₋₆ alkyl, optionally substituted C₂₋₆ alkenyl, optionally substituted C₂₋₆ alkynyl, optionally substituted C₃₋₇ carbocyclyl, optionally substituted C₆₋₁₀ aryl, optionally substituted 5-10 membered heteroaryl, and optionally substituted 3-10 membered heterocyclyl as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy (C₁-C₆)alkyl (i.e., ether), aryloxy, sulfhydryl(mercapto), halo (C₁-C₆)alkyl (e.g., —CF₃), halo(C₁-C₆)alkoxy (e.g., —OCF₃), C₁-C₆ alkylthio, arylthio, amino, amino (C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

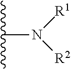

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

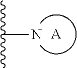

where ring A is a heteroaryl ring containing the depicted nitrogen.

Similarly, when two “adjacent” R groups are said to form a ring “together with the atom to which they are attached,” it is meant that the collective unit of the atoms, intervening bonds, and the two R groups are the recited ring. For example, when the following substructure is present:

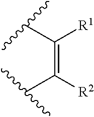

and R¹ and R² are defined as selected from the group consisting of hydrogen and alkyl, or R¹ and R² together with the atoms to which they are attached form an aryl or carbocylyl, it is meant that R¹ and R² can be selected from hydrogen or alkyl, or alternatively, the substructure has structure:

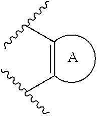

where A is an aryl ring or a carbocylyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

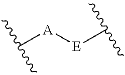

includes the substituent being oriented such that the A is attached at the leftmost attachment point of the molecule as well as the case in which A is attached at the rightmost attachment point of the molecule.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

“Solvate” refers to the compound formed by the interaction of a solvent and a compound described herein, a metabolite, or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

### Crystalline Forms of the Compound of Formula (I)

Disclosed herein are the crystalline forms of the compound of Formula (I). Unless otherwise stated, the X-ray powder diffraction data provided herein was determined using a Cu Kα radiation source.

In some embodiments, the crystalline form of the compound of Formula (I) may take the shape of rods or elongated plates. In some embodiments, the crystalline form of the compound of Formula (I) may be a hydrated crystalline material containing a variable amount of water, ranging between one and three molar equivalents.

### Form A

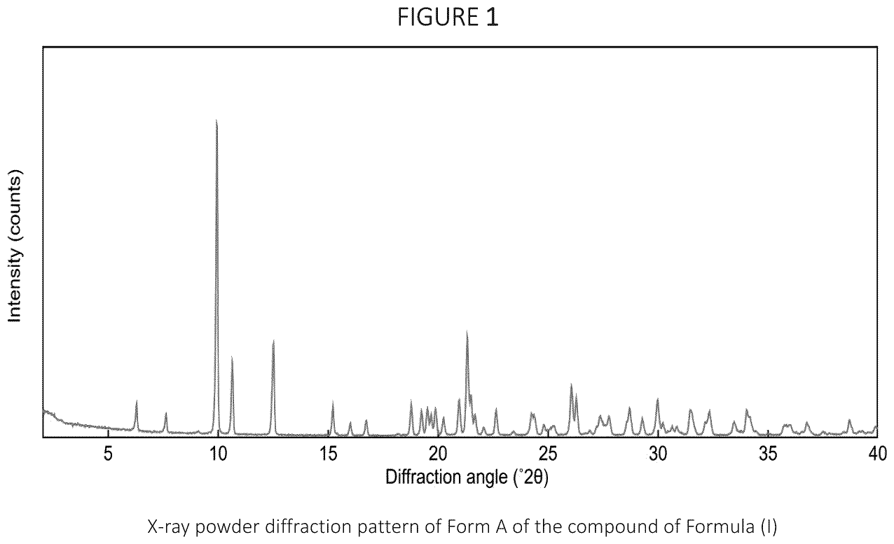

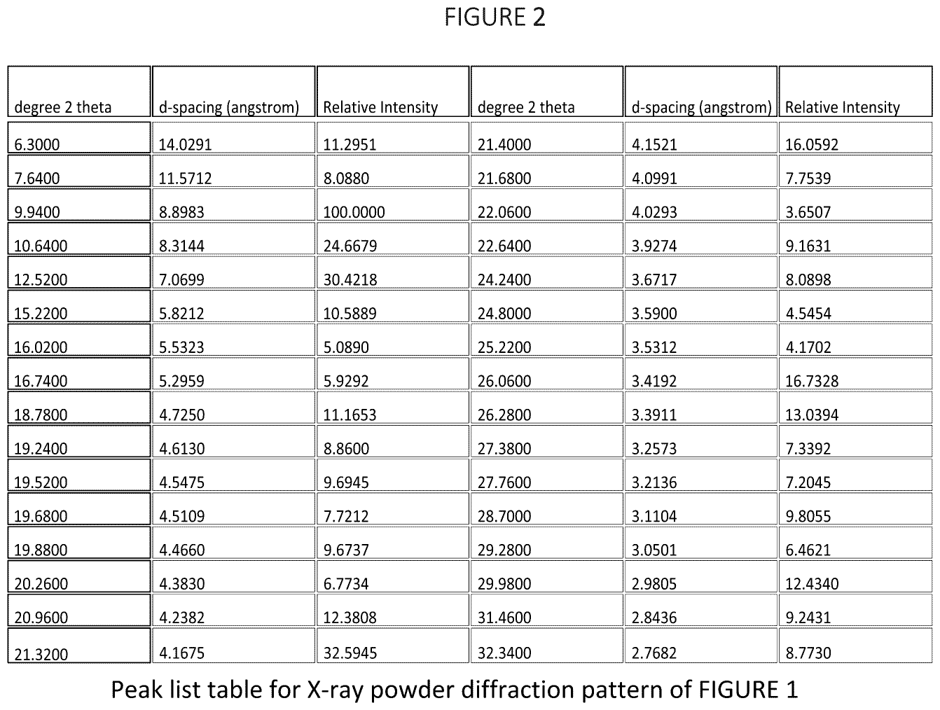

Crystalline Form A of the compound of Formula (I) was characterized using various techniques, which are described in further detail in the experimental methods section. FIG. 1 shows the X-ray powder diffraction (XRPD) pattern of Form A, and FIG. 2 shows the peak data for FIG. 1. Form A, which may be obtained by the methods disclosed herein, exhibits prominent peaks at approximately 6.3, 9.9, 10.6, 12.5, 15.2, 18.8, 20.3, 21.0, 21.3, 21.4, 26.1, 26.3, and 30.0 degrees 2θ. Form A contains unique peaks at 6.3, 9.9, 10.6, and 12.5 degrees 2θ. In some embodiments, Form A is characterized by having at least one characteristic peak (e.g., one, two, three, four, five, six, seven, eight, nine, ten or eleven characteristic peaks) selected from approximately 6.3, 9.9, 10.6, 12.5, 15.2, 18.8, 20.3, 21.0, 21.3, 21.4, 26.1, 26.3, and 30.0 degrees 2θ. In some embodiments, Form A is characterized by having at least three characteristic peaks selected from approximately 6.3, 9.9, 10.6, 12.5, 15.2, 18.8, 20.3, 21.0, 21.3, 21.4, 26.1, 26.3, and 30.0 degrees 2θ.

As is well understood in the art, because of the experimental variability when X-ray diffraction patterns are measured on different instruments, the peak positions are assumed to be equal if the two theta (20) values agree to within a certain degree of variability. For example, the United States Pharmacopeia states that if the angular setting of the 10 strongest diffraction peaks agree to within ±0.2 degrees with that of a reference material, and the relative intensities of the peaks do not vary by more than 20%, the identity is confirmed. Accordingly, in some embodiments, peak positions recited herein include variability within ±0.5 degrees 2θ. In other embodiments, peak positions recited herein include variability within ±0.2 degrees 2θ. As disclosed herein, the term “approximately” when referring to values of 2θ is defined as ±0.5 degrees 2θ.

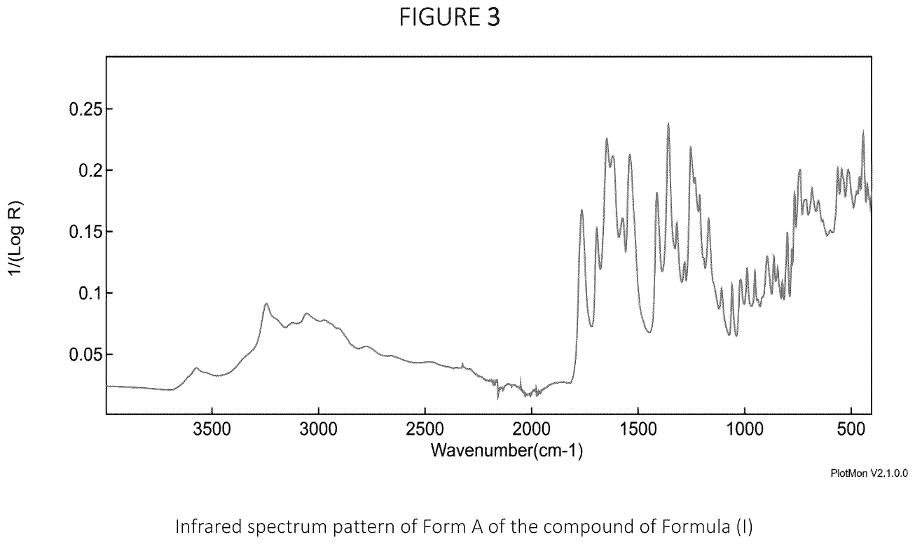

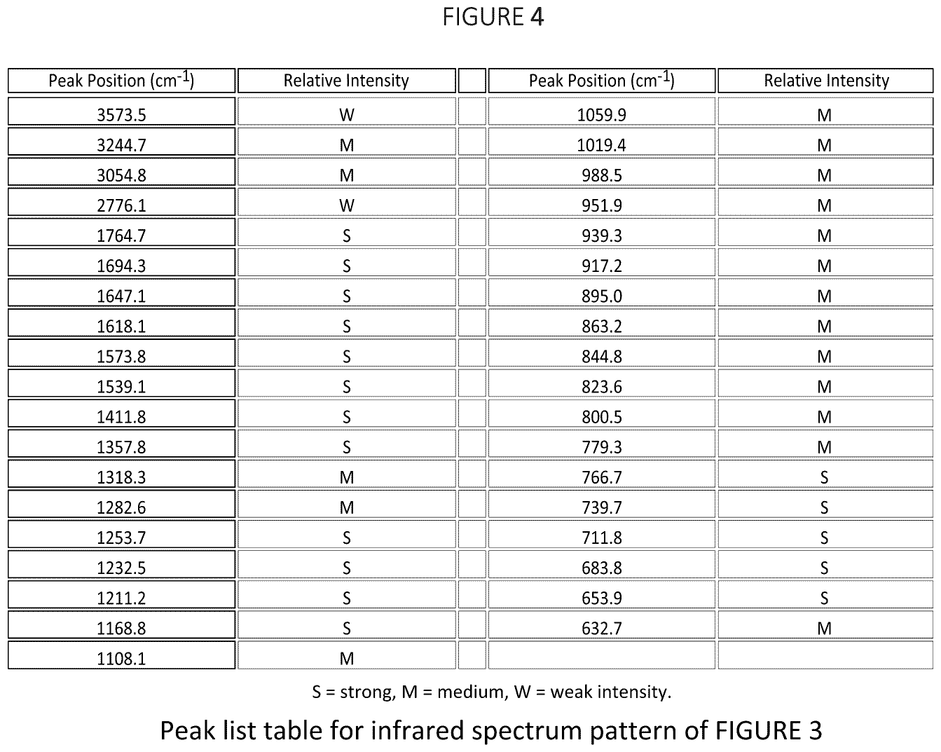

FIG. 3 shows the infrared spectrum of Form A, and FIG. 4 shows the peak data for FIG. 3. Form A exhibits prominent peaks at 3574, 1765, 1694, 1647, 1618, 1574, 1539, 1412, 1358, 1254, 1233, 1211, 1169, 767, 740, 712, 684, and 654 cm⁻¹. Form A contains unique peaks at 3574, 1765, 1694, 1647, 1574, 1539, and 1169 cm⁻¹.

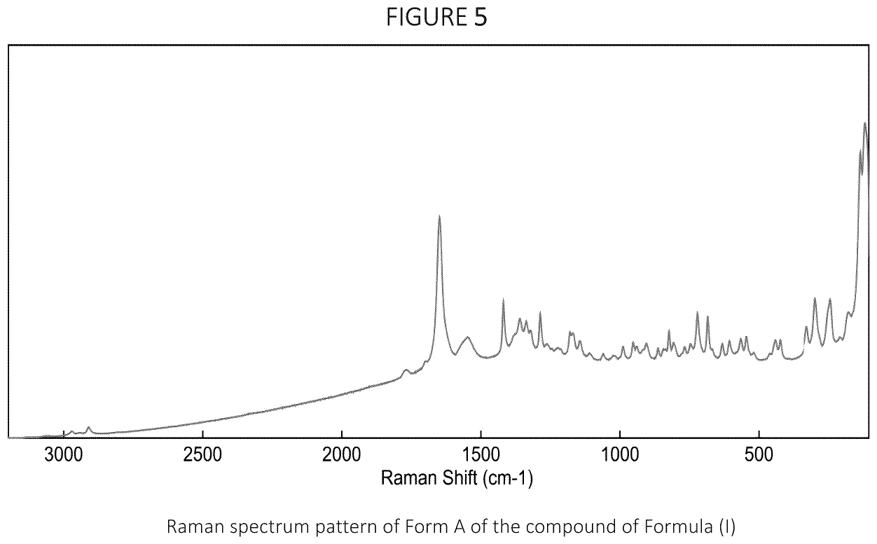

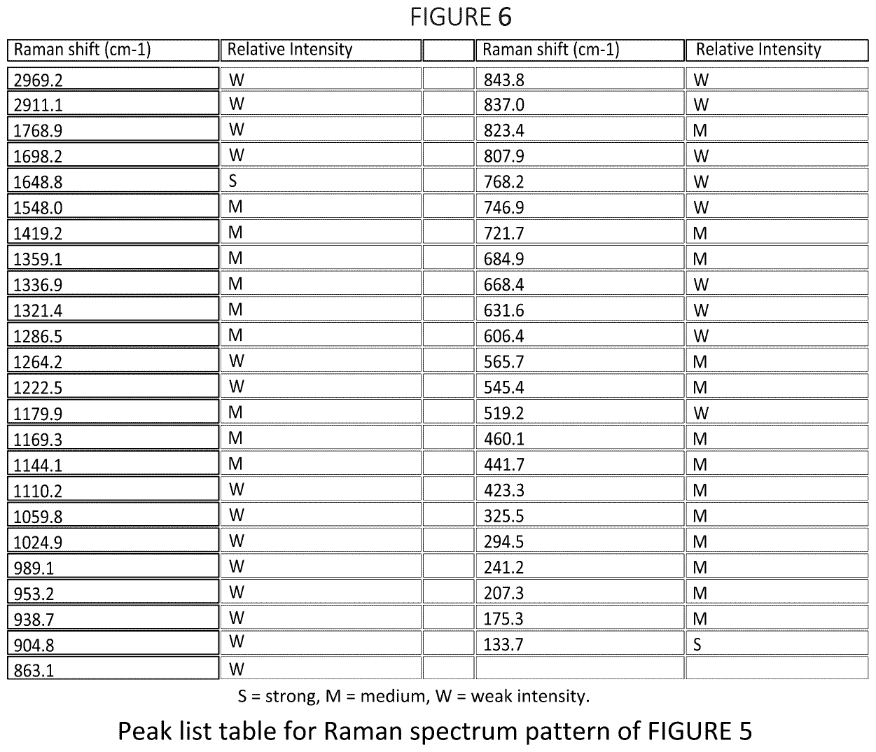

FIG. 5 shows the Raman spectrum for Form A, and FIG. 6 shows the peak data for FIG. 5. Form A exhibits prominent peaks at 1649, 1548, 1419, 1359, 1337, 1321, 1287, 1180, 1170, 1144, 823, 722, 685, 566, 545, 442, 423, 326, 295, 241, 207, 175, and 134 cm⁻¹. Form A contains unique peaks at 1649, 1419, 722, and 134 cm⁻¹.

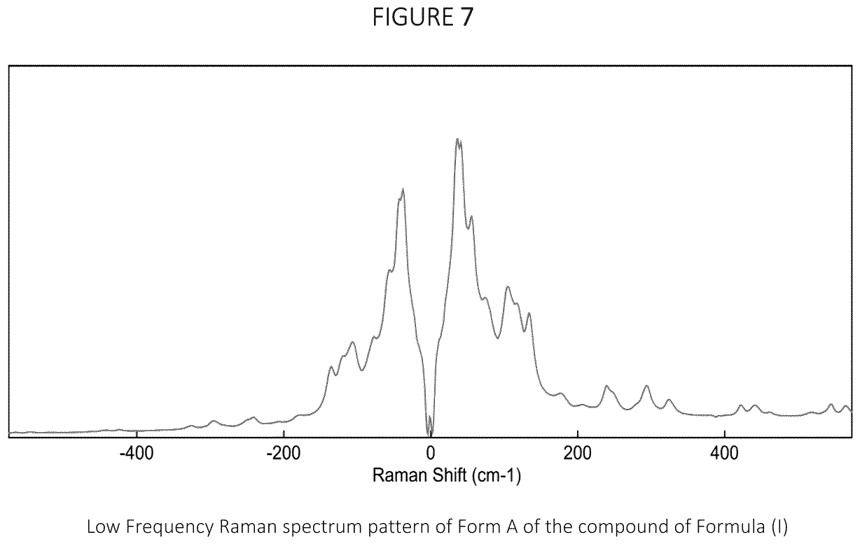

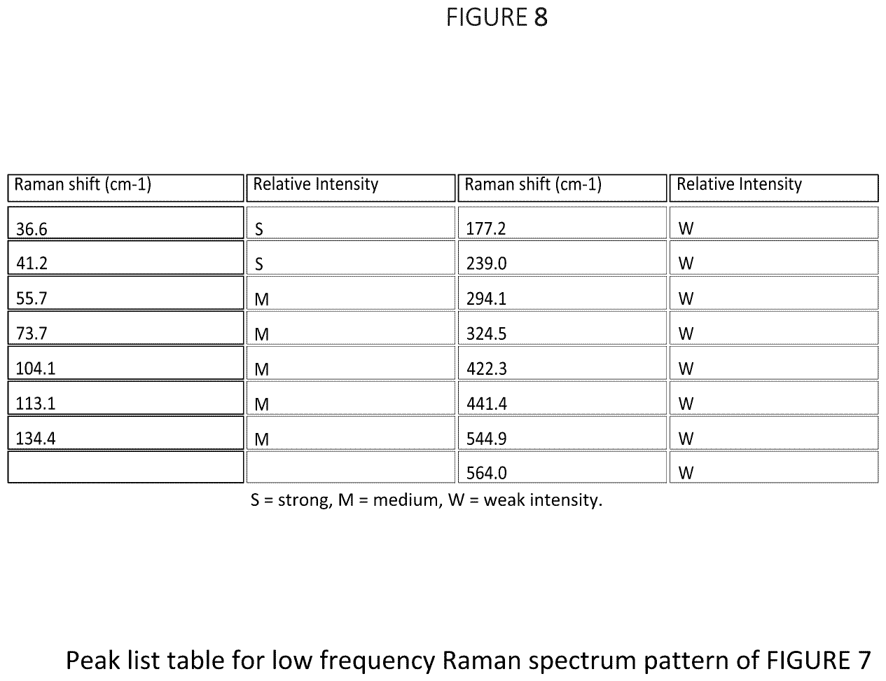

FIG. 7 shows the low frequency Raman spectrum for Form A, and FIG. 8 shows the peak data for FIG. 7. Form A exhibits prominent peaks at 36.6, 41.2, 55.7, 73.7, 104.1, 113.1, and 134.4 cm⁻¹. Form A has unique peaks at 36.6, 41.2, 55.7, and 134.4 cm⁻¹.

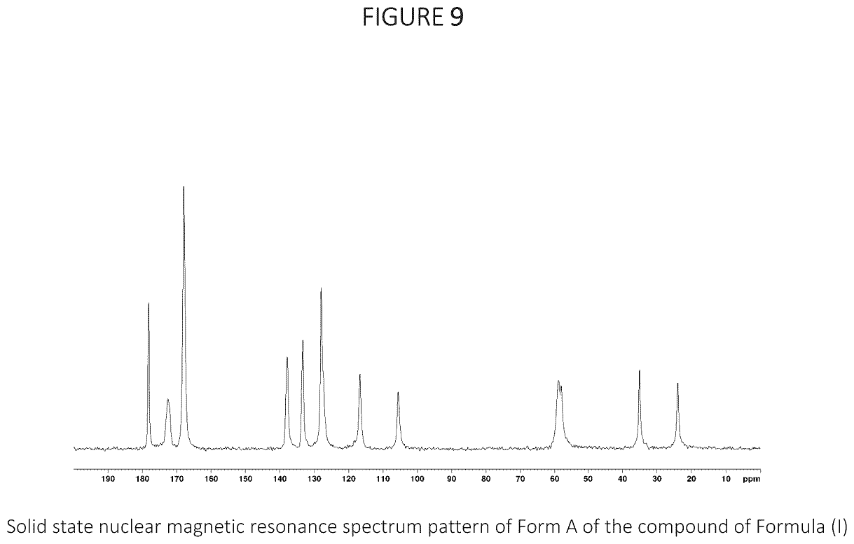

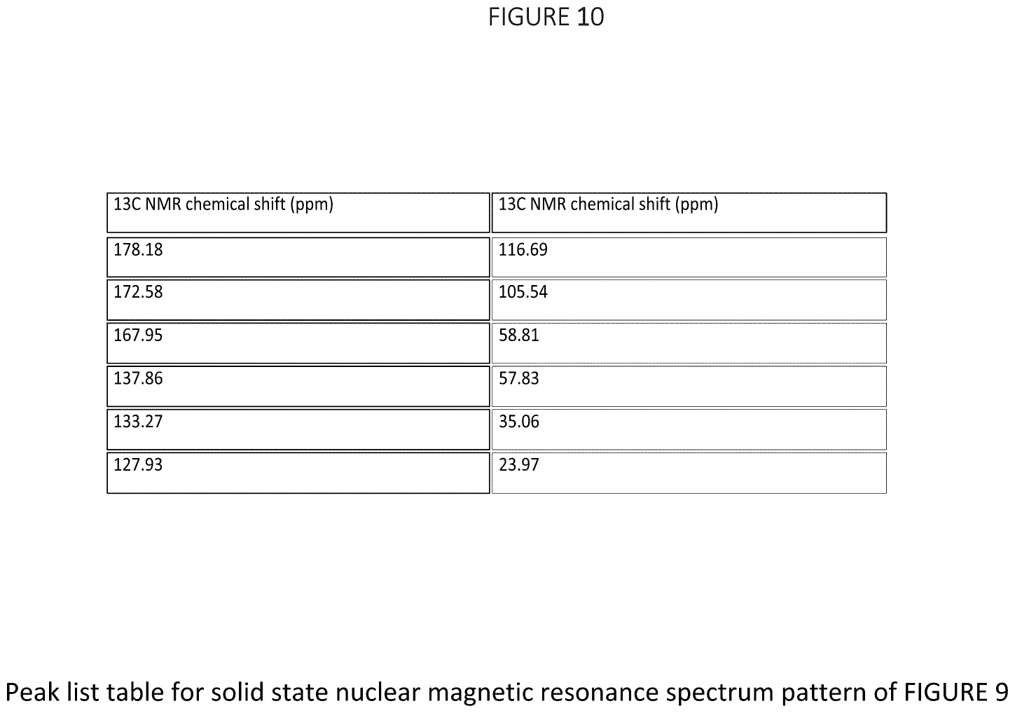

FIG. 9 shows the solid state nuclear magnetic resonance spectrum for Form A, and FIG. 10 shows the peak data for FIG. 9. Form A exhibits prominent peaks at 178.18, 172.58, 167.95, 137.86, 133.27, 127.93, 116.69, 105.54, 58.81, 57.83, 35.06, and 23.97 ppm. Form A has unique peaks at 178.18, 167.95, 137.86, 127.93, and 116.69 ppm.

### Form B

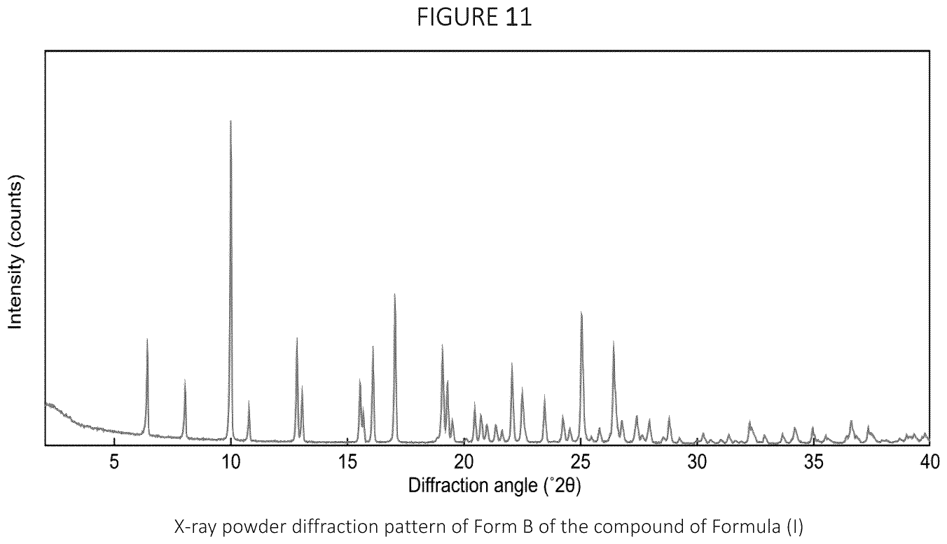

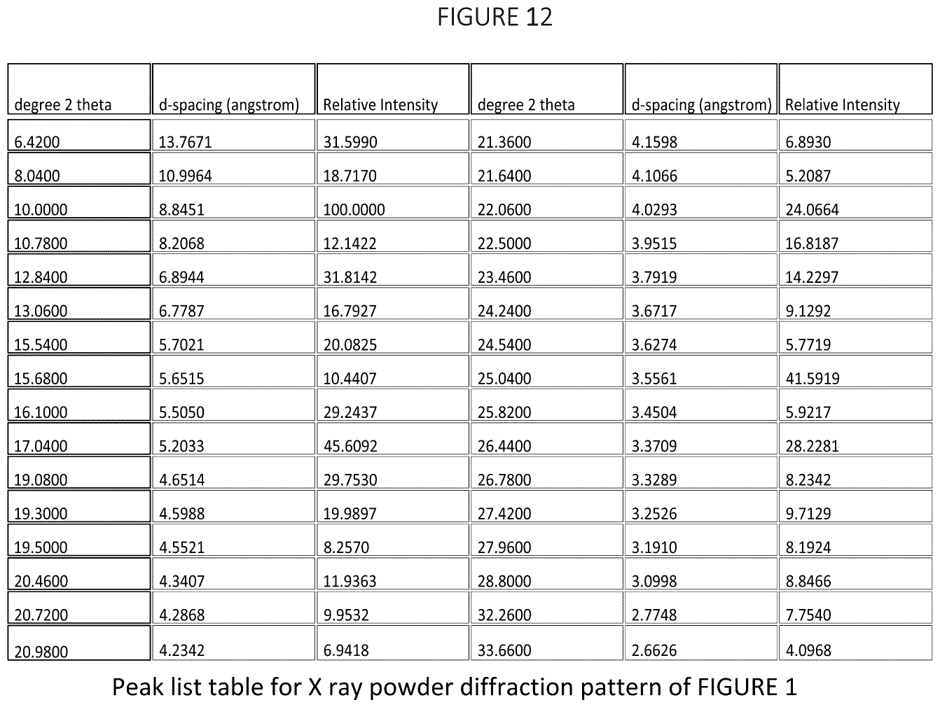

Crystalline Form B of the compound of Formula (I) was characterized using various techniques, which are described in further detail in the experimental methods section. FIG. 11 shows the X-ray powder diffraction (XRPD) pattern of Form B, and FIG. 12 shows the peak data for FIG. 11. Form B, which may be obtained by the methods disclosed herein, exhibits prominent peaks at approximately 6.4, 8.0, 10.0, 12.8, 13.1, 15.5, 16.1, 17.0, 19.1, 19.3, 20.5, 22.1, 22.5, 23.5, 25.0, and 26.4 degrees 2θ. Form B contains unique peaks at 8.4, 12.8, 13.1, and 17.0 degrees 2θ. In some embodiments, Form B is characterized by having at least one characteristic peak (e.g., one, two, three, four, five, six, seven, eight, nine, ten or eleven characteristic peaks) selected from approximately 6.4, 8.0, 10.0, 12.8, 13.1, 15.5, 16.1, 17.0, 19.1, 19.3, 20.5, 22.1, 22.5, 23.5, 25.0, and 26.4 degrees 2θ. In some embodiments, Form B is characterized by having at least three characteristic peaks selected from approximately 6.4, 8.0, 10.0, 12.8, 13.1, 15.5, 16.1, 17.0, 19.1, 19.3, 20.5, 22.1, 22.5, 23.5, 25.0, and 26.4 degrees 2θ. In some embodiments, under long-term ambient temperature and humidity conditions, Form B exhibits enhanced stability.

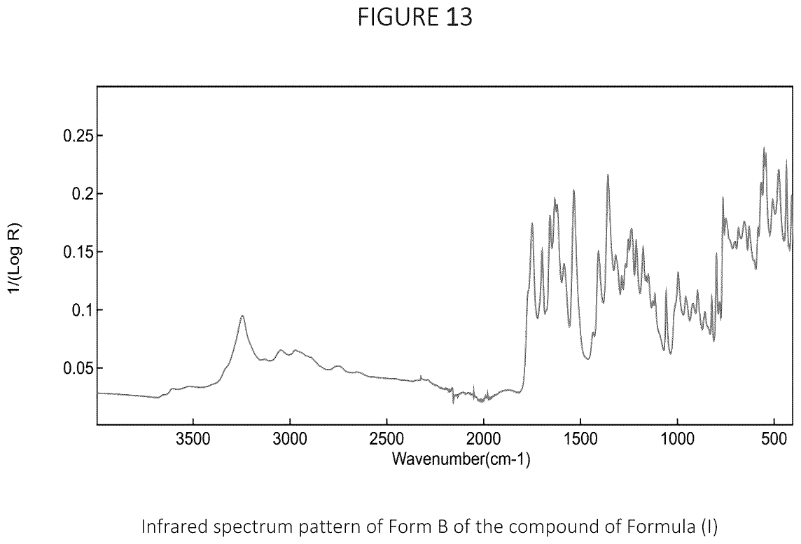

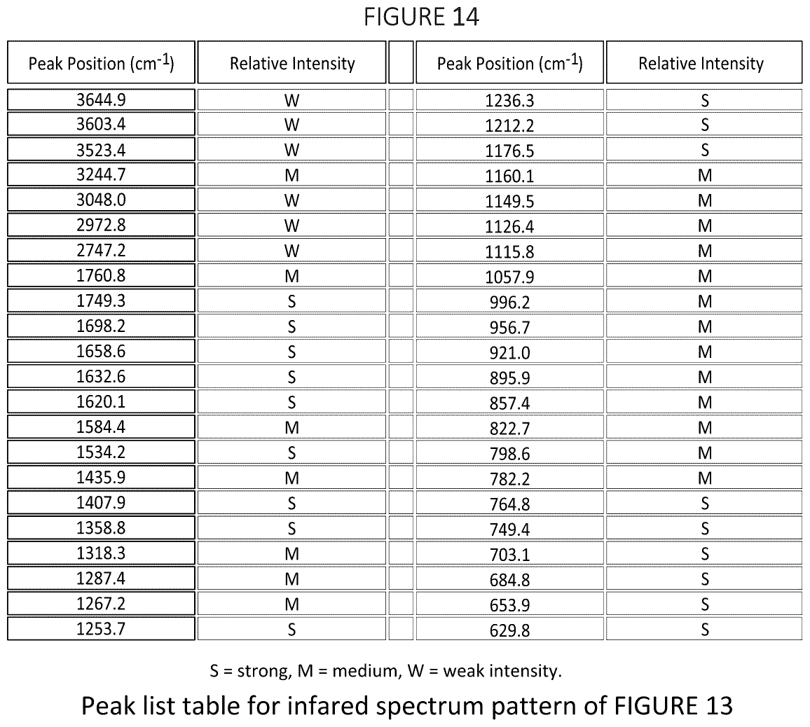

FIG. 13 shows the infrared spectrum of Form B, and FIG. 14 shows the peak data for FIG. 13. Form B exhibits prominent peaks at 3245, 1761, 1749, 1698, 1659, 1633, 1630, 1584, 1534, 1436, 1408, 1359, 1318, 1287, 1267, 1253, 1236, 1212, 1177, 1160, 1150, 1126, 1116, 1058, 996, 957, 921, 856, 823, 799, 782, 765, 749, 703, 685, 654, and 630 cm⁻¹. Form B contains unique peaks at 1749, 1698, 1659, 1633, 1584, 1532, and 1177 cm⁻¹.

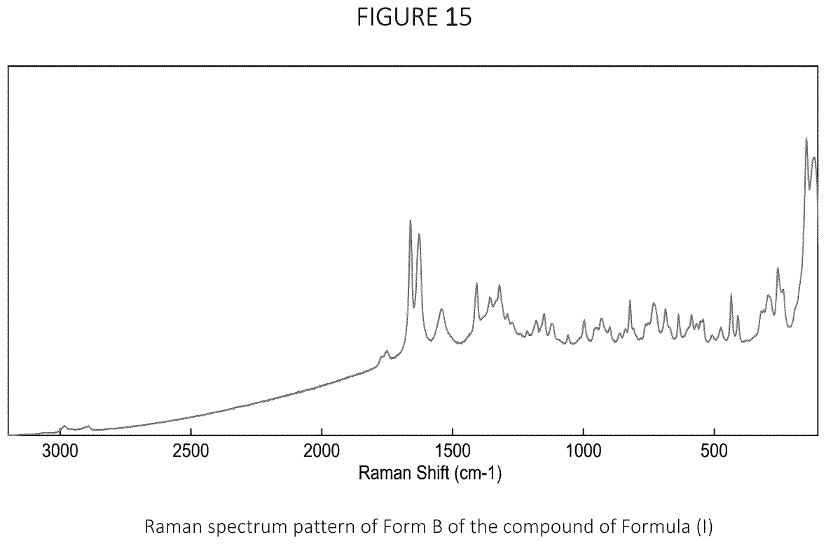

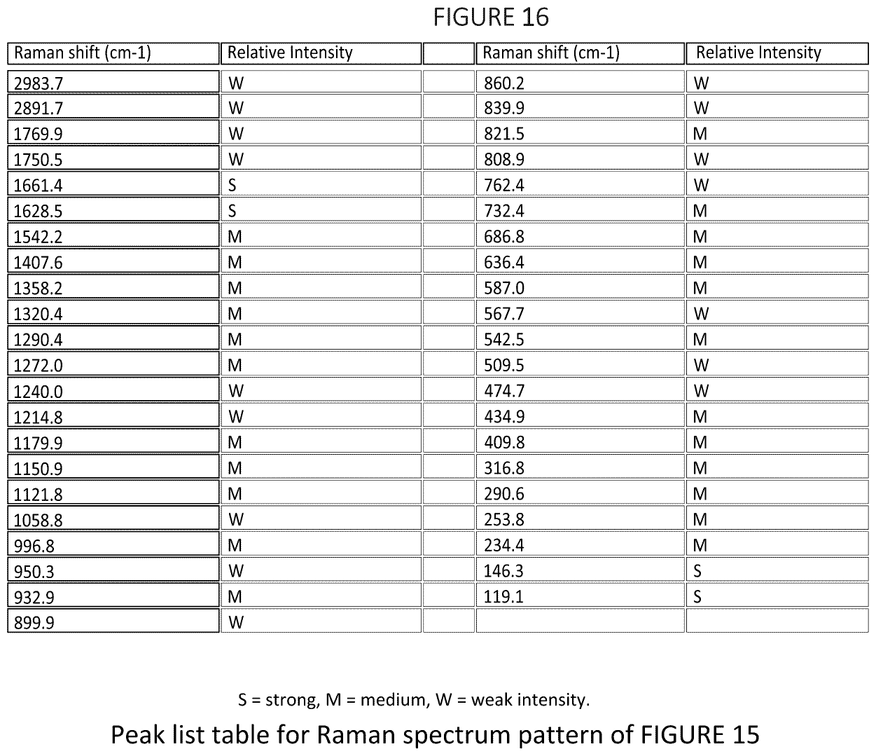

FIG. 15 shows the Raman spectrum for Form B, and FIG. 16 shows the peak data for FIG. 15. Form B exhibits prominent peaks at 1661, 1629, 1542, 1408, 1358, 1320, 1290, 1272, 1180, 1151, 1129, 997, 933, 822, 732, 687, 636, 587, 543, 435, 410, 317, 291, 254, 234, 146, and 119 cm⁻¹. Form B contains unique peaks at 1661, 1629, and 1408 cm⁻¹.

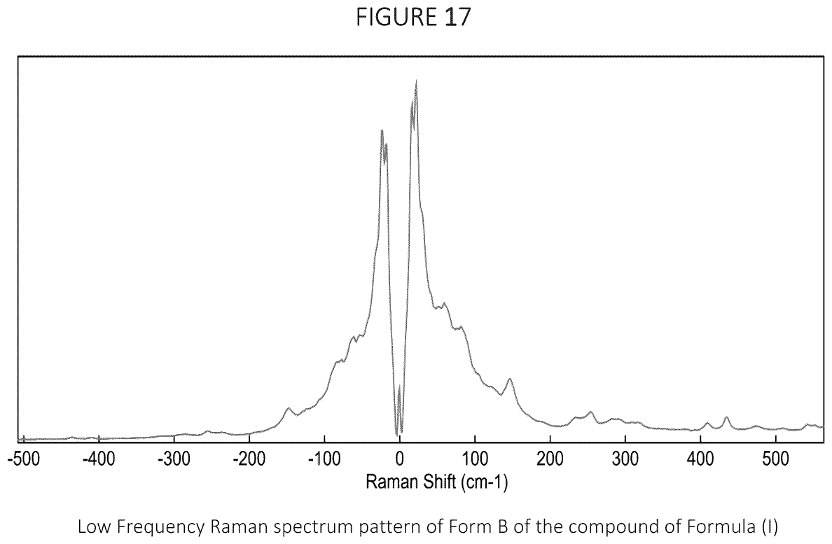

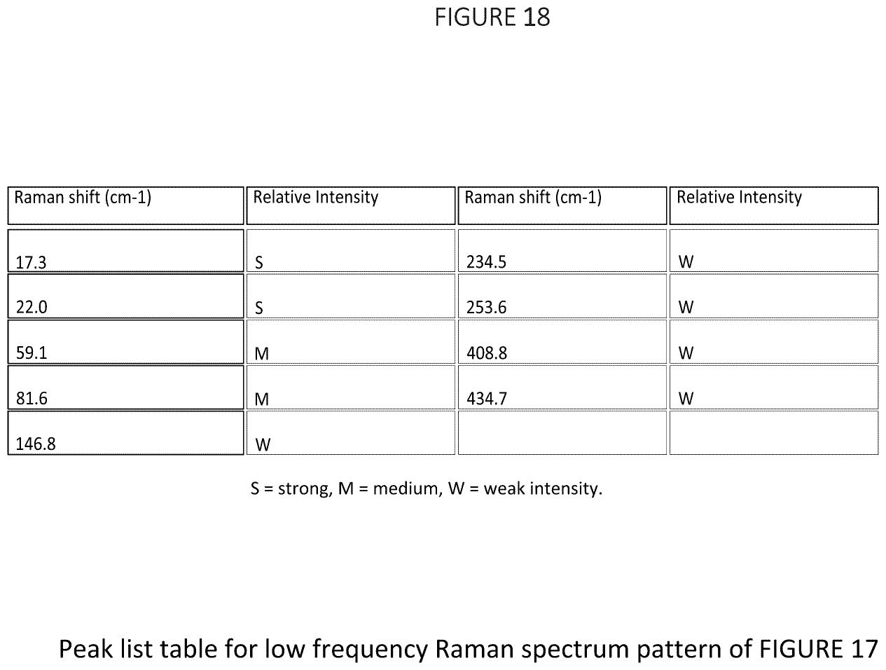

FIG. 17 shows the low frequency Raman spectrum for Form B, and FIG. 18 shows the peak data for FIG. 17. Form B exhibits prominent peaks at 17.3, 22.0, 59.1, 81.6, and 146.8 cm⁻¹. Form A contains unique peaks at 17.3, 22.0, and 146.8 cm⁻¹.

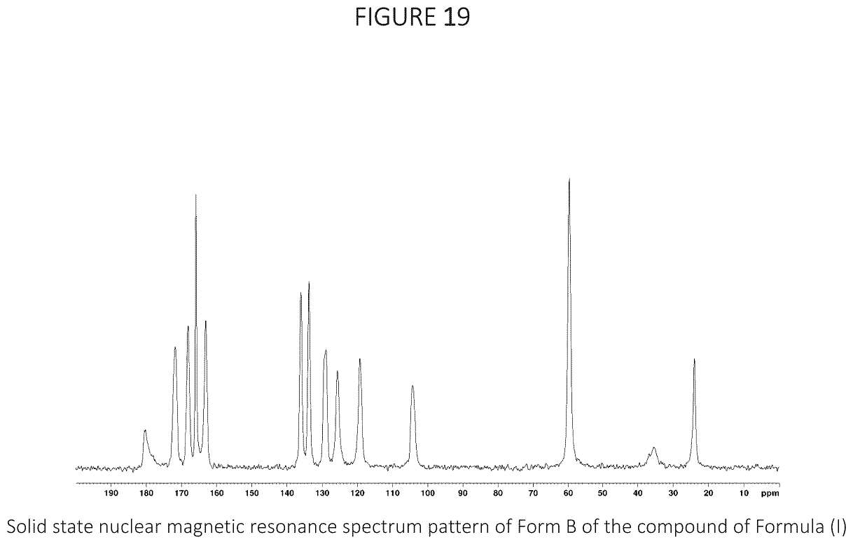

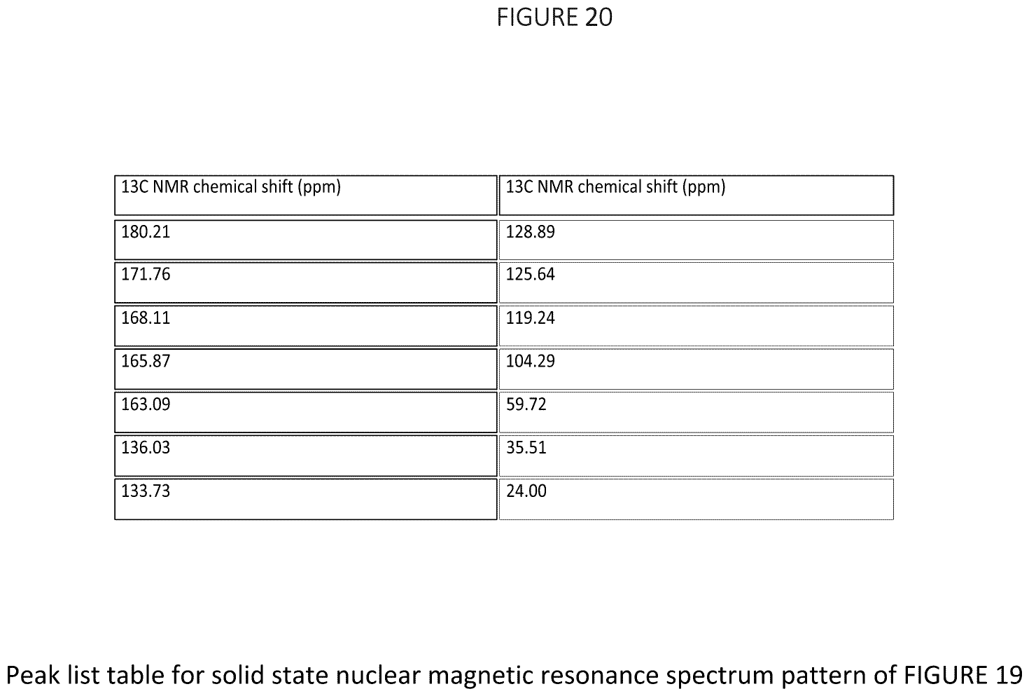

FIG. 19 shows the solid state nuclear magnetic resonance spectrum for Form B, and FIG. 20 shows the peak data for FIG. 19. Form B exhibits prominent peaks at 180.21, 171.76, 168.11, 165.87, 163.09, 136.03, 133.73, 128.69, 125.64, 119.24, 104.29, 59.72, 35.51, and 24.00 ppm. Form B has unique peaks at 171.76, 168.11, 165.87, 163.09, 136.03, 133.73, 125.64, and 119.24 ppm.

### Methods of Crystallizing the Compound of Formula (I)

Some embodiments include methods of crystalizing the compound of Formula (I). Crystalline forms of the compound of Formula (I) may generally be obtained or produced by crystallizing the compound of Formula (I) under controlled conditions. In some embodiments, the method may produce a single crystalline form. In some embodiments, the method may produce a mixture of two crystalline forms.

In some embodiments, the method may comprise dissolving a crude form of the compound of Formula (I) in demineralized water and acetone. In some embodiments, the mixture may be cooled to a temperature between 20° C. and 25° C. In some embodiments, while the mixture is stirring, sodium bicarbonate and water may be added to the mixture. In some embodiments, the pH may be balanced between 6.5 and 6.9. In some embodiments, aqueous sulfuric acid may be added to the solution. In some embodiments, the sulfuric acid may be a 15% aqueous solution. In some embodiments, the pH may be balanced between 6.5 and 6.7. In some embodiments, aluminum oxide, Norit charcoal, sodium dithionite, and disodium EDTA may be added. In some embodiments, the solution may be stirred for a time between 20 minutes and 25 minutes while maintaining a pH between 6.5 and 6.7 using aqueous sulfuric acid. In some embodiments, after the mixture has stirred, the mixture may be filtered and washed with demineralized water. In some embodiments, the filtered solution may be added over time to a vessel containing water, malic acid, and aqueous sulfuric acid. In some embodiments, the filtered solution may be added over a 20 minute to 30 minute period. In some embodiments, the temperature of the mixture may be held between 20° C. and 25° C. In some embodiments, after the solution has been stirred for a period of time ranging from 50 minutes to 70 minutes, the pH of the solution may be adjusted over time by adding potassium carbonate and demineralized water. In some embodiments, the solution may be stirred for a period of time between 20 minutes and 25 minutes. In some embodiments, the pH adjustment with potassium carbonate and demineralized water may be between a pH of 2.9 and 3.1. In some embodiments, the pH adjustment may be held at a temperature between 20° C. and 25° C. In some embodiments, the solution may be stirred for a period of time ranging between 2 hours and 2.5 hours, after which the solution may be filtered and washed with demineralized water. In some embodiments, the residue after filtration may be dried in a vacuum oven. In certain embodiments, the oven may be set at 25° C., 30° C., 35° C., or 40° C.

In some embodiments, the desired form of Formula (I) can be prepared by controlled drying. Depending on the drying method, a different form of the compound of Formula (I) can be prioritized.

### Preparation Methods of Form A

Crystalline Form A may be made by placing the compound of Formula (I) in a vessel with HPLC grade water and stirring for a period of time ranging from 12 hours to 24 hours. In some embodiments, the mixture may then be vacuum filtered and dried for a time period ranging from 60 minutes to 120 minutes in ambient conditions.

In some embodiments, the compound of Formula (I) may be placed in a vessel with HPLC grade water and stirred at a reduced temperature for a time period ranging from 1 day to 3 days. In certain embodiments, the reduced temperature may be −15° C., −10° C., −5° C., 0° C., or 5° C., or a range between any two of these values. In some embodiments, the mixture may be centrifuged and the liquid layer may be decanted. In some embodiments, the residue may then be air dried.

In some embodiments, crystalline Form A may be obtained by drying the compound of Formula (I) under vacuum. In some embodiments, the drying does not include flowing a gas, such as nitrogen, over the compound. In some embodiments, drying under vacuum is conducted until a desired Karl Fischer (KF) water content is achieved. In various embodiments, the target KF water content is from about 6% to about 14%, about 6% to about 12%, about 8% to about 12%, about 11% to about 12%, 6% to about 10%, about 7.5% to about 9.5%, about 8% to about 9%, about 6.5% to about 7.5%, about 8.6%, about 11.6%, or about 7%. In various embodiments, drying under vacuum is conducted for about 2 hours to about 18 hours, about 4 hours to about 12 hours, about 3 hours to about 10 hours, for about 4 hours to about 8 hours, for about 4 hours to about 6 hours, for about 6 hours to about 10 hours, or for about 6 hours to about 8 hours.

### Preparation Methods of Form B

Crystalline Form B may be made by placing the compound of Formula (I) in a vessel with methanol and stirred for a time period ranging from 5 days to 15 days. In some embodiments, the solution may then be vacuum filtered and the residue may be recovered.

In some embodiments, the compound of Formula (I) may be added to a vessel with phosphorus pentoxide for a time period ranging from 3 days to 5 days, at the end of which the crystalline product may be recovered.

In some embodiments, crystalline Form B may be obtained by drying the compound of Formula (I) under vacuum in the presence of a flow of a gas, such as nitrogen. In various embodiments, the drying is conducted for at least 8 hours, at least 10 hours, at least 12 hours, from about 8 hours to about 24 hours, from about 8 hours, to about 18 hours, from about 8 hours, to about 16 hours, or from about 8 hours to about 12 hours. In some embodiments, drying under vacuum is conducted until a desired Karl Fischer (KF) water content is achieved. In various embodiments, the target KF water content is less than about 6%, less than about 5%, less than about 4%, less than about 3%, from about 1% to about 5%, from about 1%, to about 3%, or about 2%. In some embodiments, after achieving an initial target KF water content, the compound is then rehydrated. In some embodiments, rehydration is conducted by exposing the compound to air. In various embodiments, the compound is rehydrated from about 2 hours to about 10 hours, from about 4 hours to about 8 hours, or about 6 hours. In various embodiments, the compound is rehydrated until a KF water content of from about 6% to about 10%, about 7.5% to about 9.5%, about 8% to about 9%, about 6.5% to about 7.5%, or about 8% is achieved.

### β-Lactamase Inhibitors

In some embodiments, the β-lactamase inhibitor for use as described herein is a compound having the structure of any one of Formulas (II)-(X):

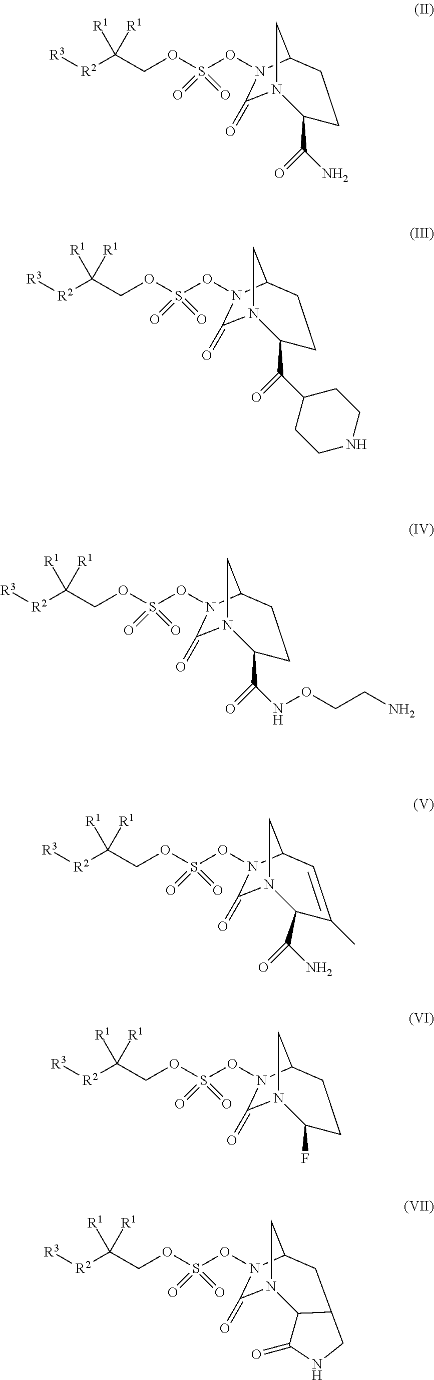

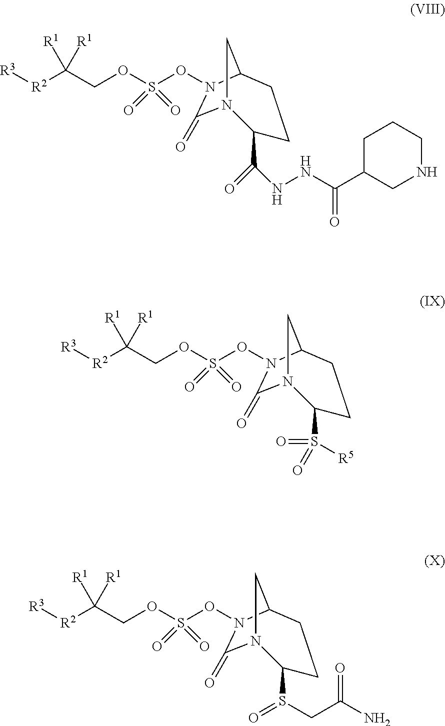

or pharmaceutically acceptable salts thereof, wherein:
each R¹ is independently a C₁₋₆ alkyl, or each R¹ together with the geminal carbon atom to which they are bonded forms an optionally substituted C₃₋₆ cycloalkyl ring or an optionally substituted 4-6 membered heterocycloalkyl ring;
R² is selected from a single bond, optionally substituted C₁₋₆ alkyl, optionally substituted 2-6 membered heteroalkyl, optionally substituted C₅₋₆ cycloalkyl, optionally substituted 5-6 membered heterocycloalkyl, optionally substituted phenyl, and optionally substituted 5-6 membered heteroaryl;
R³ is selected from C₁₋₆ alkyl, —O—C(O)—R⁴, —S—C(O)—R⁴, —NH—C(O)—R⁴, —O—C(O)—O—R⁴, —S(O)—O—R⁴, —NH—C(O)—O—R⁴, —(O)—O—R⁴, —C(O)—S—R⁴, —C(O)—NH—R⁴, —O—(O)—O—R⁴, —O—C(O)—S—R⁴, —O—C(O)—NH—R⁴, —S—S—R⁴, —S—R⁴, —NH—R⁴, and —CH(—NH₂)—R⁴);
R⁴ is selected from hydrogen, optionally substituted C₁₋₈ alkyl, optionally substituted 2-8 membered heteroalkyl, optionally substituted C₅₋₈ cycloalkyl, optionally substituted 5-8 membered heterocycloalkyl, optionally substituted C₅₋₁₀ cycloalkylalkyl, optionally substituted 5-8 membered heterocycloalkyl-C₁₋₃-alkyl, optionally substituted phenyl, optionally substituted 5-8 membered heteroaryl, optionally substituted C₇₋₁₀ arylalkyl, and optionally substituted 5-8 membered heteroaryl-C₁₋₃-alkyl;
R⁵ is selected from the group consisting of C₁₋₆ alkyl, —NR⁶R⁷, —CH₂C(O)NH₂, and

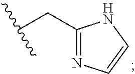 [SMILES: *Cc1ncc[nH]1]

and
R⁶ and R⁷ are independently selected from the group consisting of H, C₁₋₆ alkyl, and —CH₂C(O)NH₂.

Compounds of Formulas (II)-(X) may be made following the procedures described in U.S. Pat. No. 10,085,999, which is incorporated herein by reference in its entirety.

In other embodiments, the β-lactamase inhibitor for use as described herein is a compound having the structure of any one of Formulas (XI)-(XVIII):

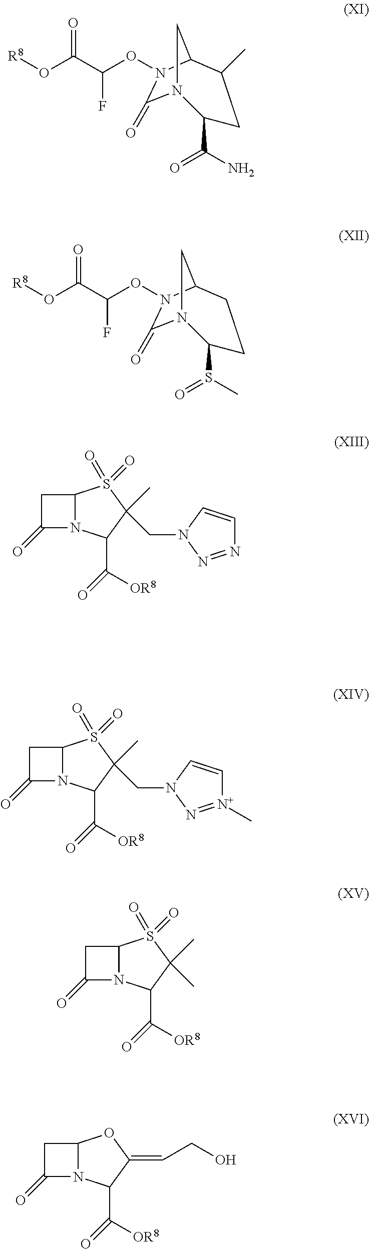

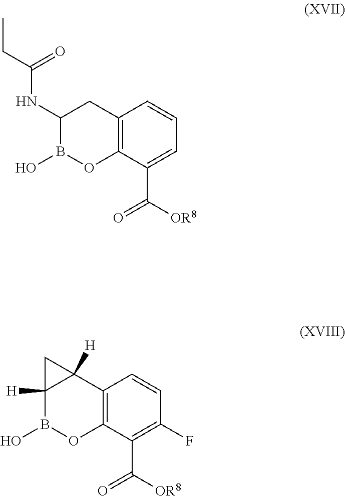

or pharmaceutically acceptable salts thereof, wherein:
R⁸ is selected from the group consisting of C₁₋₉ alkyl, —CR¹⁰R¹¹OC(O)C₁₋₉alkyl, —CR¹⁰R¹¹OC(O)C₃₋₇carbocyclyl, —CR¹⁰R¹¹OC(O)(3 to 7 membered heterocyclyl), —CR¹⁰R¹¹OC(O)C₂₋₈alkoxyalkyl, —CR¹⁰R¹¹OC(O)OC₁₋₉alkyl, —CR¹⁰R¹¹OC(O)OC₃₋₇carbocyclyl, —CR¹⁰R¹¹OC(O)O(3 to 7 membered heterocyclyl), —CR¹⁰R¹¹OC(O)OC₂₋₈alkoxyalkyl, —CR¹⁰R¹¹OC(O)C₆₋₁₀aryl, —CR¹⁰R¹¹OC(O)OC₆₋₁₀aryl, —CR¹⁰R¹¹C(O)NR¹³R¹⁴, —CR¹⁰R¹¹OC(O)O(CH₂)₁₋₃C(O)NR¹³R¹⁴, —CR¹⁰R¹¹OC(O)O(CH₂)₂₋₃OC(O)C₁₋₄alkyl, —CR¹⁰R¹¹OC(O)O(CH₂)₁₋₃C(O)OC₁₋₄alkyl, —CR¹⁰R¹¹OC(O)(CH₂)₁₋₃OC(O)C₁₋₄alkyl, and

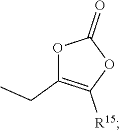

each R¹⁰ and R¹¹ is independently selected from the group consisting of H, optionally substituted C₁₋₄alkyl, optionally substituted C₃₋₇ carbocyclyl, optionally substituted 3-10 membered heterocyclyl, optionally substituted C₆₋₁₀aryl, and optionally substituted 5-10 membered heteroaryl;
each R¹³ and R¹⁴ is independently selected from the group consisting of H, optionally substituted C₁₋₆alkyl, optionally substituted C₃₋₇ carbocyclyl, optionally substituted 3-10 membered heterocyclyl, optionally substituted C₆₋₁₀aryl, and optionally substituted 5-10 membered heteroaryl; and
R¹⁵ is optionally substituted C₁₋₆ alkyl.

Compounds of Formulas (XI)-(XVIII) may be made using the procedures described in PCT Publication Nos. WO 2019/093450 or WO 2018/005662 (both of which are incorporated herein by reference in their entirety), or any other procedures for forming esters known in the art.

In some embodiments, the β-lactamase inhibitor for use as described herein is selected from the group consisting of:

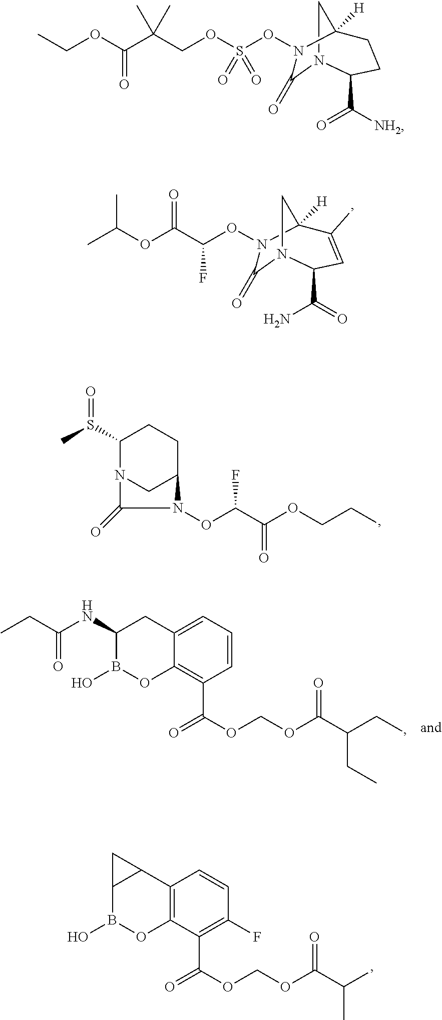

or pharmaceutically acceptable salts thereof. These compounds and their synthesis are described in U.S. Pat. No. 10,085,999, U.S Application Publication No. 2019/0202832, PCT Publication No. WO2019/093450, PCT Publication No. WO2018/005662, and PCT Application No. PCT/US2021/022799, all of which are incorporated herein by reference in their entirety.

### Administration and Pharmaceutical Compositions

The crystalline forms of the compound of Formula (I) can be provided in a pharmaceutical composition. The pharmaceutical compositions provided herein include therapeutically effective amounts of the compound of Formula (I). In some embodiments, pharmaceutical compositions may contain a pharmaceutically acceptable excipient. In some embodiments, pharmaceutical compositions may contain a β-lactamase inhibitor. The pharmaceutical compositions provided herein may be useful in the treatment of bacterial infections or bacterially related diseases or disorders.

In some embodiments, the total amount of the compound of Formula (I) in the composition may include at least about 50% by weight of a crystalline form of the compound of Formula (I). In some embodiments, the total amount of compound of Formula (I) in the composition may include at least about 85% by weight of a crystalline form of the compound of Formula (I). In some embodiments, the total amount of compound of Formula (I) in the composition may include at least about 90% by weight of a crystalline form of the compound of Formula (I). In some embodiments, the total amount of compound of Formula (I) in the composition may consist essentially entirely a crystalline form of the compound of Formula (I). In some embodiments, the total amount of compound of Formula (I) in the composition may consist essentially entirely of a crystalline form of compound of Formula (I). In these embodiments, any remaining portion of the compound of Formula (I) in the composition may be another crystalline form or in non-crystalline form.

Administration of the compounds disclosed herein can be via any of the accepted modes of administration including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds disclosed can be formulated into pharmaceutical compositions, either individually or together. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) one or more compounds disclosed herein; and (b) a pharmaceutically acceptable carrier, diluent, excipient or combination thereof.

In addition to the selected compound useful as described above, some embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents. In liquid oral solutions, a crystalline form of Formula (I) described herein may be dissolved in a suitable solvent.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above. In liquid solutions, a crystalline form of Formula (I) described herein may be dissolved in a suitable solvent.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses. In liquid ophthlmic solutions, a crystalline form of Formula (I) described herein may be dissolved in a suitable solvent.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol. In liquid intravenous solutions, a crystalline form of Formula (I) described herein may be dissolved in a suitable solvent.

The compositions for intravenous administration may be provided to caregivers in the form of one or more solids that are reconstituted with a suitable diluent such as sterile water, saline or dextrose in water shortly prior to administration. For example, a crystalline form of Formula (I) described herein may be provided in a container for reconstitution. In other embodiments, the compositions are provided in solution ready to administer parenterally. For example, a crystalline form of Formula (I) may be dissolved in a solvent prior to providing the formulation to a caregiver. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

In some embodiment, the compound of Formula (I) and a β-lactamase inhibitor may be co-administered. By “co-administered,” it is meant that the two agents are administered so as to have a biological effect at the same time, regardless of when or how they are actually administered. In some embodiments, the two agents may be found in the patient's bloodstream at the same time. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered intravenous (i.v.).

The effective amount of a compound provided herein can be determined by one of ordinary skill in the art, and includes exemplary dosage amounts for a mammal of from about 0.05 to 100 mg/kg of body weight of active compound per day, which can be administered in a single dose or in the form of individual divided doses, such as from 1 to 4 times per day. It will be understood that the specific dose level and frequency of dosage for any particular subject can be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. In some embodiments, the daily dosage of a compound provided herein can be varied over a wide range from about or 0.01 to about or 1000 mg per adult human per day. For example, dosages can range from about or 0.1 to about or 800 mg/day. In some embodiments, the dosage can range from 0.2 mg to 200 mg per day. In some embodiments, the dosage can range from 0.5 mg to 100 mg per day. In some embodiments, the daily dosage can be 0.1 mg, 0.25 mg, 0.5 mg, 0.75 mg, 1 mg, 2 mg, 3 mg, 5 mg, 7.5 mg, 10 mg, 50 mg, 100 mg, 200 mg, 400 mg, 600 mg, or 800 mg, or a range between any two of these values. For oral administration, the compositions can be provided in the form of unit dosages such as tablets or capsules or liquids including from about or 0.01 to about or 1000 mg, such as for example, 0.01, 0.05, 0.075, 0.1, 0.25, 0.5, 0.75, 1, 2, 2.5, 3, 4, 5, 6, 7, 8, 9, 10, 15, 20, 25, 50, 75, 100, 125, 150, 175, 180, 190, 200, 225, 250, 300, 400, 500, 750, 800, 850, 900, 950 and 1000 milligrams, or a range between any two of these values, of the active ingredient for the symptomatic adjustment of the dosage to the subject to be treated. In some embodiments, the compositions can be provided in the form of unit dosages such as tablets or capsules or liquids including from about or 0.01 to about or 1000 μg, such as for example, 0.01, 0.05, 0.075, 0.1, 0.25, 0.5, 0.75, 1, 2.5, 3, 4, 5, 6, 7, 8, 9, 10, 15, 20, 25, 50, 75, 100, 125, 150, 175, 180, 190, 200, 225, 250, 300, 350, 400, 450, 500, 550, 600, 650, 700, 750, 800, 850, 900, 950 and 1000 micrograms, or a range between any two of these values, of the active ingredient for the symptomatic adjustment of the dosage to the subject to be treated.

### Methods of Treatment

Methods of use of the compounds and compositions provided herein also are provided. The methods include in vitro and in vivo uses of the compounds and compositions for eliminating bacterial activity and for treatment, prevention, or amelioration of one or more symptoms of diseases or disorder that are alleviated through the elimination of bacterial activity, or in which bacterial activity is implicated. In certain embodiments, provided herein are methods of treating a subject by administering a compound provided herein. In certain embodiments, such subject exhibits symptoms or signs of a bacterially mediated condition. In certain embodiments, a subject is treated prophylactically to reduce or prevent the occurrence of a condition. Some embodiments include a method of treating a bacterial infection by administering the compound of Formula (I) to a subject in need thereof. Some embodiments include administering the compound of Formula (I) to a subject to treat a condition including, but not limited to, acute bacterial exacerbations of chronic bronchitis; acute bacterial otitis media; pharyngitis; tonsilitis; pneumonia; urinary tract infection; enteritis; or gastroenteritis.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

Bacterial infections that can be treated with the compounds, compositions and methods described herein can comprise a wide spectrum of bacteria. Example organisms include gram-positive bacteria, gram-negative bacteria, aerobic and anaerobic bacteria, such as Staphylococcus, Lactobacillus, Streptococcus, Sarcina, Escherichia, Enterobacter, Klebsiella, Pseudomonas, Acinetobacter, Mycobacterium, Proteus, Campylobacter, Citrobacter, Nisseria, Baccillus, Bacteroides, Peptococcus, Clostridium, Salmonella, Shigella, Serratia, Haemophilus, Brucella and other organisms.

More examples of bacterial infections include Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

### EXAMPLES

Additional embodiments are disclosed in further detail in the following examples, which are not in any way intended to limit the scope of the claims.

### Example 1: Preparation of Ceftibuten Hydrates from Crude Formula (I)

In a reaction vessel are charged 400 mL of demineralized water, 37.0 g crude Ceftibuten and 190 mL acetone at 20-25° C. To this mixture is added with stirring a solution of 22.1 g NaHCO₃ and 252.2 mL of demineralized water, keeping the pH at 6.5-6.9. The pH is then adjusted to 6.5-6.7 using 15% aqueous H₂SO₄. To the resulting solution are charged 13.8 g aluminum oxide, 5 g Norit charcoal, 1.5 g Na₂S₂O₄ and 0.25 g disodium EDTA. The mixture is stirred for 20-25 min maintaining pH at 6.5-6.7 with 15% aqueous H₂SO₄, and is then filtered and washed with 200 mL of demineralized water. The filtrate is added over 20-30 min to a second vessel containing 630 mL water, 81 g malic acid and 175 g 15% aqueous H₂SO₄, maintaining the temperature at 20-25° C. After stirring for 50-70 min, the pH is adjusted to 2.9-3.1 over 20-25 min at 20-25° C. with a solution constituted by 30 g of K₂CO₃ and 70 g of demineralized water. This mixture is stirred for 2-2.5 hours, after which it is filtered and washed 5 times with demineralized water (250 mL each). The wet product is dried at 30° C. in a vacuum oven. Depending on the extent of drying, the resulting product may be Form A, Form B, or a mixture of Forms A and B. Three batches prepared by this procedure are shown in Table 1 below. Upon storage/shipping at ambient temperature and humidity, Batch 1 converted from a mixture to all Form B. This indicates increased stability of Form B.

TABLE 1
Cefitbuten hydrate moisture content and form.

| Batch No. | Moisture Content | Form |
|---|---|---|
| 1 | 9.44% | A + B |
| 2 | 8.60% | B |
| 3 | 11.68% | A |

### Example 2: Preparation of Form A

Method 1: Form A can be prepared from a mixture of Form A and B. The mixed forms were converted to pure Form A as follows: 192 mg of ceftibuten Form A/B was placed in a glass vial and 5 mL of HPLC grade water was added. The slurry formed was magnetically stirred at ambient temperature for 16 hours and vacuum filtered. The wet cake was allowed to dry on a filter paper at ambient conditions for 90 minutes to yield 156 mg of ceftibuten Form A.

Method 2:40 mg of ceftibuten Form A/B was placed in a glass vial and 0.5 mL of HPLC grade water was added. The slurry formed was magnetically stirred at 0° C. for 2 days. The vial containing the slurry was centrifuged and the water was decanted to obtain ceftibuten Form A as a wet cake which was air dried.

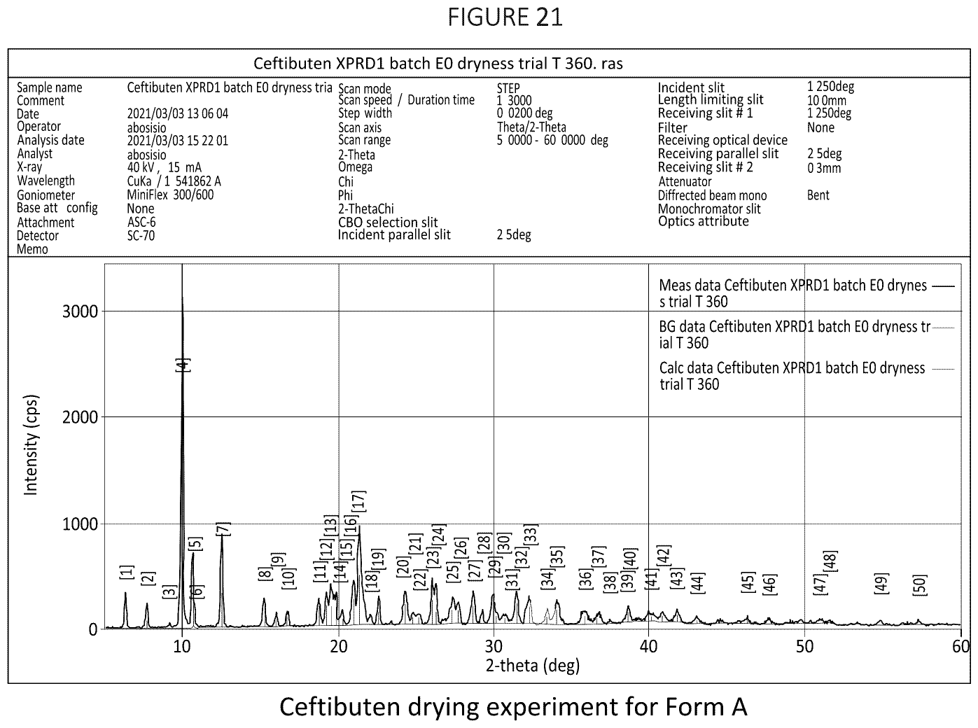

Method 3: Putative ceftibuten Form A was dried in a rotative dryer to KF of 8.6% to give ceftibuten Form A. Further drying to KF of 7% indicated persistence of Form A. FIG. 21 contains the X-ray powder diffraction pattern after this procedure, demonstrating Form A.

### Example 3: Preparation of Form B

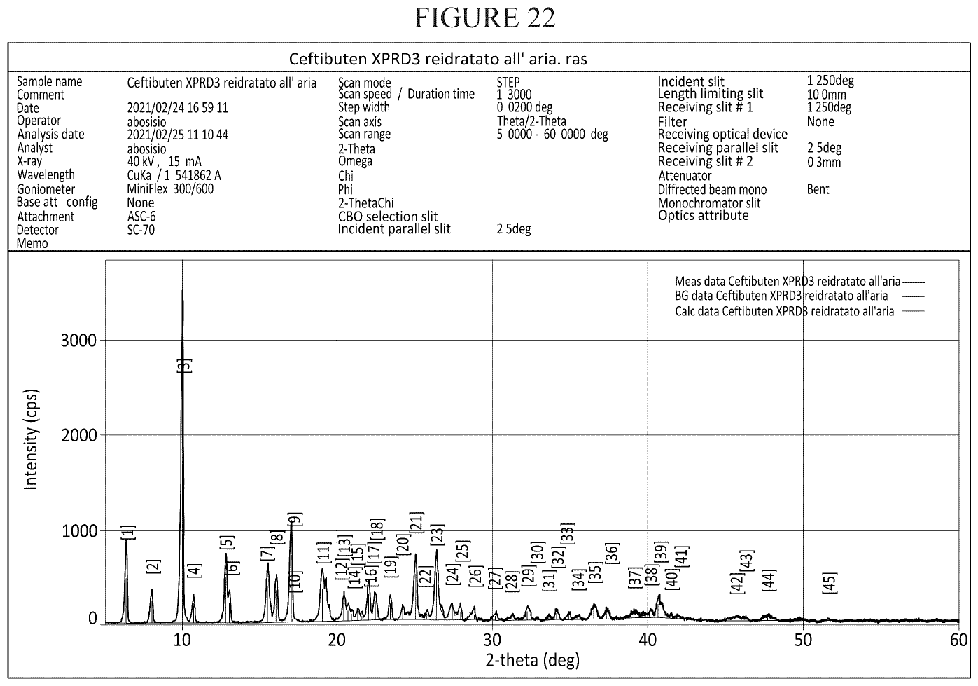

Method 1: About 20 mg of ceftibuten Form A was placed in a glass vial and 1 mL of methanol was added. The slurry formed was stirred at ambient temperature for 11 days and vacuum filtered to give off-white colored solid ceftibuten Form B.
Method 2: About 20 mg of ceftibuten Form A in an open vial was placed in a sealed jar containing phosphorus pentoxide for 4 days to give ceftibuten Form B.
Method 3: About 20 mg of ceftibuten Form A in an open vial was placed in a sealed jar containing phosphorus pentoxide for 4 days to give ceftibuten Form B.
Method 4: A sample of ceftibuten Form A was dried overnight under vacuum and 30° C. N₂ flow to KF of 2% and then rehydrated by air exposure (6 hours) to KF of 8% to give ceftibuten Form B. FIG. 22 contains the X-ray powder diffraction pattern after this procedure, demonstrating Form B.

### Example 4: Physical Stability

Form A of the compound of Formula (I) may be moderately hygroscopic and may be stable under a range of humidity conditions. In some embodiments, Form A of the compound of Formula (I) may contain between two to three molar equivalents of water. Samples of Form A of the compound of Formula (I) were exposed to several relative humidity conditions ranging from 15% to 100% relative humidity for a period of 14 days, after which an X-ray powder diffraction was taken.

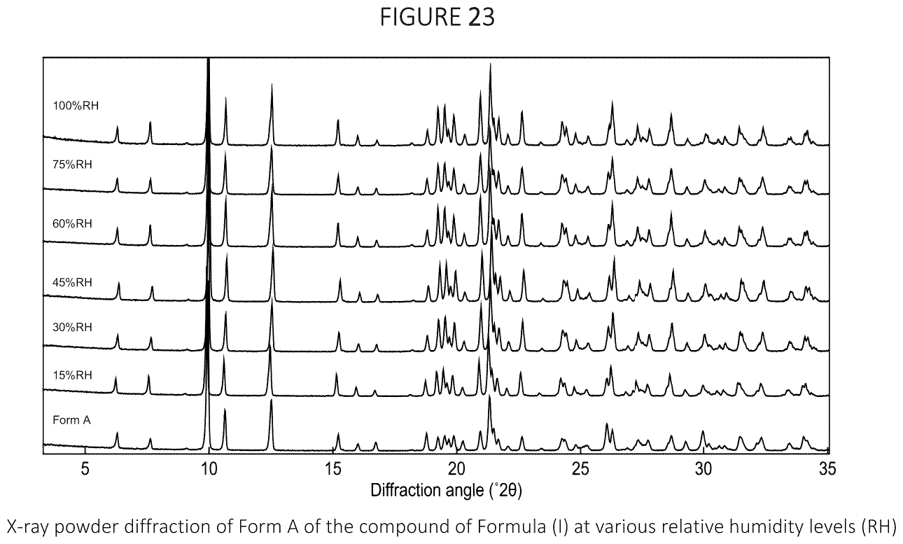

In one embodiment, samples Form A of the compound of Formula (I) were exposed to several relative humidity conditions, including 15% relative humidity, 30% relative humidity, 45% relative humidity, 60% relative humidity, 75% relative humidity, and 100% relative humidity for a period of 14 days, after which an X-ray powder diffraction was taken. FIG. 23, the X-ray powder diffraction taken after the relative humidity experiment, shows peaks that are a match for Form A of the compound of Formula (I).

Form B of the compound of Formula (I) may be moderately hygroscopic. In some embodiments, Form B of the compound of Formula (I) may contain between two to three molar equivalents of water. In some embodiments, under extremely dry conditions such as 0% relative humidity, Form A of the compound of Formula (I) may convert to Form B of the compound of Formula (I).

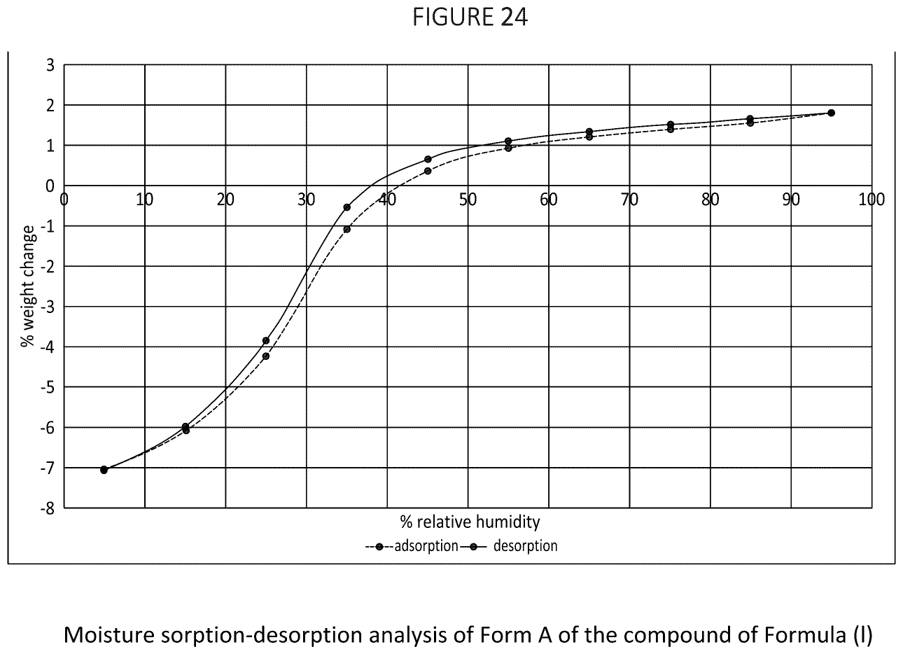

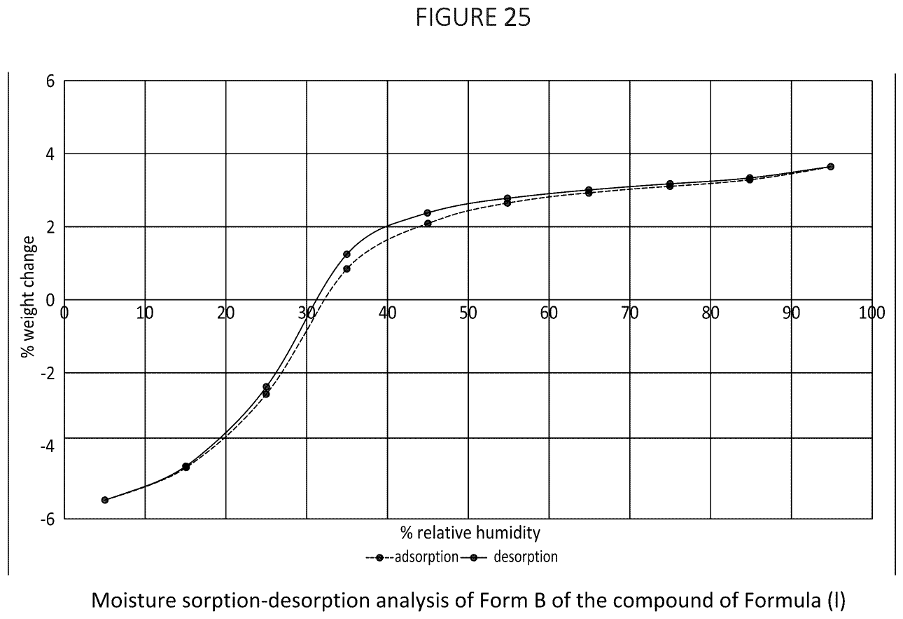

In one embodiment, moisture sorption-desorption analyses were taken after running the moisture sorption-desorption experiments. In the moisture sorption-desorption analysis for Form A of the compound of Formula (I), seen in FIG. 24, Form A of the compound of Formula (I) is shown to convert to Form B of the compound of Formula (I). In the moisture sorption-desorption analysis for Form B of the compound of Formula (I), seen in FIG. 25, Form B of the compound of Formula (I) is shown to remain stable and unchanged.

### Example 5: Differential Scanning Calorimetry (DSC)

DSC analysis was carried out using a TA Instruments Q2500 Discovery Series instrument. The instrument temperature calibration was performed using indium. The DSC cell was kept under a nitrogen purge of ˜50 mL per minute during the analysis. The sample was placed in a standard, crimped, aluminum pan and was heated from approximately 25° C. to 350° C. at a rate of 10° C. per minute.

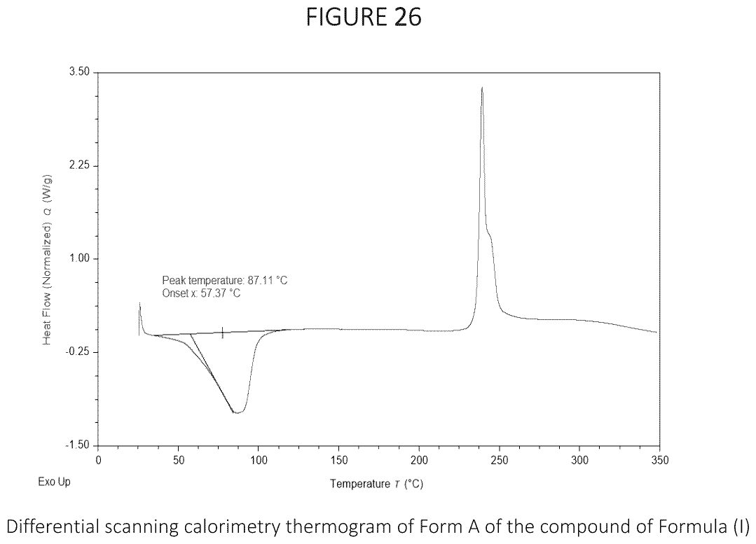

Form A of the compound of Formula (I) dehydrates during heating at approximately 100° C., and decomposes between 220° C. and 270° C. without exhibiting a melting event. FIG. 26, a differential scanning calorimetry thermogram of Form A of the compound of Formula (I), shows the dehydration and decomposition.

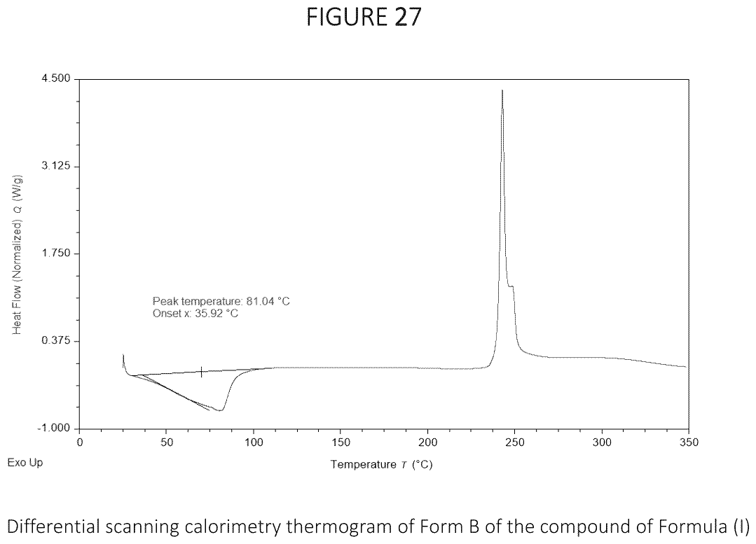

Form B of the compound of Formula (I) dehydrates during heating at approximately 100° C., and decomposes between 220° C. and 270° C. without exhibiting a melting event. FIG. 27, a differential scanning calorimetry thermogram of Form B of the compound of Formula (I), shows the dehydration and decomposition of Form B.

### Example 6: X-Ray Powder Diffraction

X-ray diffraction was used to characterize the compound of Formula (I). A Rigaku Smart-Lab X-ray diffraction system was configured for reflection Bragg-Brentano geometry using a line source X-ray beam. The x-ray source was a Cu Long Fine Focus tube that may be operated at 40 kV and 44 mA. That source provides an incident beam profile at the sample that changes from a narrow line at high angles to a broad rectangle at low angles. Beam conditioning slits may be used on the line X-ray source to ensure that the maximum beam size is less than 10 mm both along the line and normal to the line. The Bragg-Brentano geometry was a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1° 2θ or less. The axial divergence of the X-ray beam is controlled by 5.0-degree Soller slits in both the incident and diffracted beam paths.

Powder samples of the compound of Formula (I) were prepared in a low background Si holder using light manual pressure to keep the sample surfaces flat and level with the reference surface of the sample holder. Each sample was analyzed from 2 to 40° 2θ using a continuous scan of 6° 2θ per minute with an effective step size of 0.02° 2θ.

The XRPD diffractogram of Form A is depicted in FIG. 1 and the resulting peak characteristics are provided in FIG. 2. The XRPD diffractogram of Form B is depicted in FIG. 3 and the resulting peak characteristics are provided in FIG. 4.

### Example 7: Dynamic Vapor Sorption (DVS) Analysis

DVS analysis was carried out TA Instruments Q5000 Dynamic Vapor Sorption analyzer. The instrument was calibrated with standard weights and a sodium bromide standard for humidity. Samples were analyzed at 25° C. with a maximum equilibration time of 60 minutes in 10% relative humidity (RH) steps from 5 to 95% RH (adsorption cycle) and from 95 to 5% RH (desorption cycle).

DVS analysis of Form A is shown in FIG. 24 and DVS analysis of Form B is shown in FIG. 25.

### Example 8: Infrared (IR) Spectroscopy

Infrared spectrum was obtained on a Nicolet 6700 FT-IR system, using a Nicolet SMART iTR attenuated total reflectance device equipped with deuterated triglycine sulfate (DTGS) detector. Analysis was performed using spectral range of 4000-400 cm⁻¹ with a spectral resolution of 2 cm⁻¹, and 128 accumulations per spectrum.

FIG. 3 shows the infrared spectrum for Form A with peaks listed in FIG. 4, and FIG. 13 shows the infrared spectrum for Form B with peaks listed in FIG. 14.

### Example 9: Raman Spectroscopy

Raman spectroscopy analysis was performed using a Renishaw inVia™ Qontor™ confocal microscope. The system interfaces a Leica DM2700M microscope to a dispersive Raman spectrometer. Test materials were placed onto a motorized XYZ stage underneath the 20× objective of the microscope. Each sample was excited by a 785 nm polarized diode laser through the microscope and the scattered light was collected along the same optical path as the incoming laser using a 180° backscattering geometry. The scattered light from the sample passed through a notch filter and a 1200 lines/mm grating where it was dispersed onto a charge-coupled detector (CCD). Each sample was analyzed using a 10 second exposure time over the spectral range from 3200 cm⁻¹ to 100 cm⁻¹ (Raman shift) with a spectral resolution of approximately 3 cm⁻¹. Eight accumulations were collected.

The Raman spectrum for Form A is depicted in FIG. 5, with peaks listed in FIG. 6. The Raman spectrum for Form B is depicted in FIG. 15, with peaks listed in FIG. 16.

### Example 10: Low Frequency (LF) Raman Spectroscopy

Low frequency Raman spectra were obtained using a Renishaw inVia Raman microscope equipped with an Ondax THz-Raman system (TR-PROBE; excitation laser 853.1 nm, notch filter). The sample powder was analyzed in the open air using the probe tip attachment. Spectra were acquired using a static scan centered at 36 cm⁻¹ to collect over the spectral range-575 to 575 cm⁻¹ with 100% power, an exposure time of one second, and 32 accumulations. The wavelength calibration was confirmed using a sulfur reference standard. Data acquisition was performed using WIRE 3.4 software.

The low-frequency Raman spectrum for Form A is depicted in FIG. 7, with peaks listed in FIG. 8. The low-frequency Raman spectrum for Form B is depicted in FIG. 17, with peaks listed in FIG. 18.

### Example 11: Karl Fischer (KF) Analyses

Karl Fischer analyses were carried out using a Mettler-Toledo C20 Coulometric KF titrator. The instrument was calibrated using a Hydranal water standard containing 1% water. The titrant was a Hydranal methanol solution.

### Example 12: ¹³C Solid-State Nuclear Magnetic Resonance (NMR) Spectroscopy

Solid-state NMR (ssNMR) spectra were measured on a Bruker Avance II 400 MHZ (9.4 T) spectrometer operating at a ¹³C Larmor frequency of 100.6 MHz. All experiments were performed using a Doty probe with 4 mm Si₃N₄ rotors. Each spectrum was processed using TopSpin v3.2 and a ¹³C chemical shift was externally referenced to the methylene signal of adamantane at 38.48 ppm on the TMS scale.

It is possible to perform the ¹³C CPMAS analysis on NMR spectrometers with different magnetic fields, such as 9.4 Tesla (100 MHz for ¹³C, 400 MHz for 1H) or higher. Number of parameters such as acquisition time, dwell time, recycle delay, spin speed, and number of scans can be modified and optimized depending on the NMR spectrometer.

The ssNMR spectrum for Form A is depicted in FIG. 9, with peaks listed in FIG. 10. The ssNMR spectrum for Form B is depicted in FIG. 19, with peaks listed in FIG. 20.

### Example 13: Spectrographic Analysis of Formulations

For solid dosage drug products containing the compound of Formula (I), the formulated products can be analyzed by XRPD to identify the crystalline form in the product. This can be achieved by deconvoluting the XRPD data obtained from the formulated product, subtracting the known excipient signals from the XRPD data of the formulated product. In addition, various chemometrics techniques can be used to identify the compound in the product, for example principal component analysis.

What is claimed is:

1. A crystalline form of a compound of Formula (I): 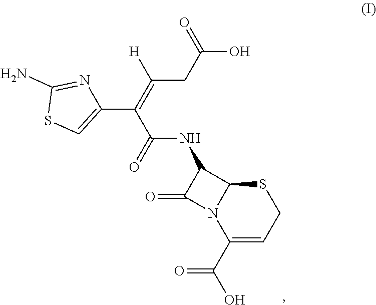
or a solvate thereof.

2. The crystalline form of claim 1, wherein the crystalline form exhibits an X-ray powder diffraction pattern comprising at least one characteristic peak, wherein said characteristic peak is selected from the group consisting of approximately 6.4, 8.0, 10.0, 12.8, 13.1, 15.5, 16.1, 17.0, 19.1, 19.3, 20.5, 22.1, 22.5, 23.5, 25.0, and 26.4 degrees 2θ.

3. The crystalline form of claim 2, wherein the crystalline form exhibits an X-ray diffraction powder diffraction pattern comprising at least three characteristic peaks, wherein said characteristic peaks are selected from a group consisting of 6.4, 8.0, 10.0, 12.8, 13.1, 15.5, 16.1, 17.0, 19.1, 19.3, 20.5, 22.1, 22.5, 23.5, 25.0, and 26.4 degrees 2θ.

4. A composition comprising a crystalline form of any one of claims 1 to 2, wherein the total weight of the compound of Formula (I) in the composition comprises greater than 50% by weight of the crystalline form.

5. The composition of any one of claims 1 to 2, wherein the total weight of the compound of Formula (I) in the composition comprises greater than 85% by weight of the crystalline form.

6. The composition of any one of claims 1 to 2, wherein the total weight of the compound of Formula (I) in the composition comprises greater than 90% by weight of the crystalline form.

7. A composition of any one of claims 1 to 6, where the crystalline form is a hydrate.

8. A method of treating a bacterial infection comprising administering to a subject in need thereof a therapeutically effective amount of the compound of any one of claims 1 to 2.

9. A method of treating a disease or disorder comprising administering to a subject in need thereof a therapeutically effective amount of the crystalline form of any one of claims 1 to 2, wherein the disease or disorder is selected from the group consisting of: acute bacterial exacerbations of chronic bronchitis; acute bacterial otitis media; pharyngitis; tonsilitis; pneumonia; urinary tract infection; enteritis; and gastroenteritis.

10. The method of claim 9, wherein the disease or disorder is selected from the group consisting of: acute bacterial exacerbations of chronic bronchitis; acute bacterial otitis media; pharyngitis; and tonsilitis.

11. The method of claim 9, wherein the method of treatment further comprises administering a β-lactamase inhibitor.

12. A pharmaceutical composition, comprising a therapeutically effective amount of the crystalline form of any one of claims 1 to 3 and one or more pharmaceutically acceptable excipients.

13. The pharmaceutical composition of claim 12, further comprising a β-lactamase inhibitor.

14. A method of preparing the crystalline form of claim 2 of 3, comprising drying the compound of Formula (I) under vacuum and nitrogen flow.

15. The method of claim 14, wherein said drying is conducted for at least 8 hours

16. The method of claim 14 or 15, wherein said drying is conducted until a Karl Fischer water content of less than 3% is achieved.

17. The method of any one of claims 14-16, comprising re-hydrating the dried compound by exposure to air.

18. The method of claim 17, wherein the exposure to air is for at least 4 hours

19. The method of claim 17 or 18, wherein the exposure to air is until a Karl Fischer water content of between 7% and 9% is achieved.

20. A method of preparing a crystalline form of a compound of Formula (I): 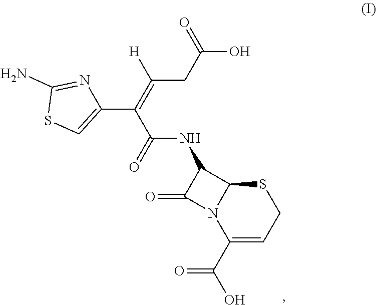
or a solvate thereof, wherein the crystalline form exhibits an X-ray diffraction powder diffraction pattern comprising at least three characteristic peaks, wherein said characteristic peaks are selected from a group consisting of 6.3, 9.9, 10.6, 12.5, 15.2, 18.8, 20.3, 21.0, 21.3, 21.4, 26.1, 26.3, and 30.0 degrees 2θ, the method comprising drying the compound of Formula (I) under vacuum.

21. The method of claim 20, wherein said drying does not comprise flowing nitrogen over the compound.

22. The method of claim 20 or 21, wherein said drying is conducted until a Karl Fischer water content of between 6% and 12% is achieved.

23. The method of any one of claims 20-22, wherein said drying is conducted until a Karl Fischer water content of between 8% and 12% is achieved.

24. The method of any one of claims 20-23, wherein said drying is conducted for between 3 hours and 8 hours.
